SEQUENCE LISTING (39 sequences)

SEQ ID NO: 1 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 2 - protein, 1380 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVY

SEQ ID NO: 3 - protein, 1629 aa, Francisella tularensis
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 4 - protein, 1122 aa, Streptococcus thermophilus
MSDLVLGLDIGIGSVGVGILNKVTGEIIHKNSRIFPAAQAENNLVRRTNRQGRRLTRRKKHRIVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNSQITDEFINRYLEILTGKRKYYHGPGNEKSRTDYGRYRTNGETLDNIFGILIGKCTFYPDEFRAAKASYTAQEFNLLNDLNNLTVPTETKKLSKEQKNQIINYVKNEKVMGPAKLFKYIAKLLSCDVADIKGHRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNPNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEEQLLDIETGELISDDEYKESVFKAPYQHFVDTLKSKEFEDSILFSYQVDSKFNRKISDATIYATRQAKVGKDKKDETYVLGKIKDIYTQDGYDAFMKIYKKDKSKFLMYRHDPQTFEKVIEPILENYPNKEMNEKGKEVPCNPFLKYKEEHGYIRKYSKKGNGPEIKSLKYYDSKLLGNPIDITPENSKNKVVLQSLKPWRTDVYFNKNTGKYEILGLKYADLQFEKKTGTYKISQEKYNGIMKEEGVDSDSEFKFTLYKNDLLLVKDTETKEQQLFRFLSRTMPNVKYYVELKPYSKDKFEKNESLIEILGSADKSGRCIKGLGKSNISIYKVRTDVLGNQHIIKNEGDKPKLDF

SEQ ID NO: 5 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 6 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 7 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 8 - protein, 1228 aa, Unknown
  Lachnospiraceae
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 9 - protein, 1052 aa, Artificial Sequence
  Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 10 - protein, 1052 aa, Artificial Sequence
  Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNNRLNKIELNMIDITYREYLENMNDKRPPRIIKTIASKTQSIKKYSTDILGNLYEVKSKKHPQIIKKG

SEQ ID NO: 11 - protein, 1052 aa, Artificial Sequence
  Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNSTRNIVELNMIDITYREYLENMNDKRPPRIIKTIASKTQSIKKYSTDILGNLYEVKSKKHPQIIKKG

SEQ ID NO: 12 - protein, 1052 aa, Artificial Sequence
  Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNSDKNNLIEVNMIDITYREYLENMNDKRPPRIIKTIASKTQSIKKYSTDILGNLYEVKSKKHPQIIKKG

SEQ ID NO: 13 - protein, 1052 aa, Artificial Sequence
  Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNSDDRNIIELNMIDITYREYLENMNDKRPPRIIKTIASKTQSIKKYSTDILGNLYEVKSKKHPQIIKKG

SEQ ID NO: 14 - protein, 1052 aa, Artificial Sequence
  Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNRDLLNRIEVNMIDITYREYLENMNDKRPPRIIKTIASKTQSIKKYSTDILGNLYEVKSKKHPQIIKKG

SEQ ID NO: 15 - protein, 1052 aa, Artificial Sequence
  Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNRDLLNLIEVNMIDITYREYLENMNDKRPPRIIKTIASKTQSIKKYSTDILGNLYEVKSKKHPQIIKKG

SEQ ID NO: 16 - DNA, 3159 bp, Staphylococcus aureus
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggcgaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggcgaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 17 - DNA, 4140 bp, Streptococcus pyogenes
atggataagaaatactcaatcggactggacatcggcactaattcggtgggatgggcagtgattaccgatgagtacaaggtcccgtcgaagaagtttaaggtgctgggaaacaccgatagacactcgatcaagaagaacctcatcggcgctctgctgtttgattccggcgagactgcggaagccacccggctgaagcgcactgcccgcagaagatacacccggcggaaaaatagaatctgctacctccaggagattttctcgaacgaaatggcaaaggtggacgattcattcttccatcgcctggaagagagcttcctggtggaagaggataagaagcatgagcgccatcctatcttcgggaacattgtggacgaagtggcctatcacgagaaatacccgactatctatcatctccgcaagaagctggtggactcaaccgataaggccgacctgaggctgatctacctggcactggcacatatgattaaattccggggccatttccttatcgagggggatctgaatccggataactccgatgtcgataagctgttcatccagctggtccagacttacaatcagctgttcgaggaaaatccgatcaatgcgtcgggcgtggacgccaaggccatcctctcggcgaggctgtcaaaaagcaggcgcctcgaaaacctgatcgcgcagctgccgggggaaaaaaagaacggcctgtttgggaatctgatcgccctctcactgggactaaccccgaatttcaagtcgaatttcgatctcgcagaagacgccaagctgcaattgtccaaggatacctacgacgatgatctggataatctactggctcagatcggagatcagtacgcggacctgttcttggccgctaagaacctctcggacgccattctgctgagcgacatcctgcgcgtcaatactgagatcaccaaagcgccactgagcgcgtcgatgattaagagatacgacgaacatcatcaggatctgactctgctgaaggccctcgtccgccagcagctcccggaaaagtacaaggagattttctttgaccagtcgaagaatggctacgcaggctatatcgacggaggcgccagccaagaagagttttacaagttcatcaagccaatcctggagaagatggacggcactgaggaactgctcgtgaagctgaaccgggaggacttgctgagaaaacagagaaccttcgacaatggaagcatcccgcaccagatccatctcggggaactgcatgctattctgagaaggcaagaggatttctacccctttctgaaggataacagagaaaagatcgaaaagattttgaccttccgcatcccatactacgtggggcctctagccaggggcaacagccggtttgcatggatgacccgcaagtcggaggaaacgatcactccgtggaacttcgaggaagtggtggacaaaggagcgtcagctcagtcctttatcgaacgcatgacgaacttcgataagaatctcccaaacgaaaaggtgcttccaaagcattcgctgctctacgaatacttcaccgtgtacaacgaactcactaaggtgaagtacgtcactgaagggatgcgcaagccggcatttctgtcgggagagcaaaagaaagcaatcgtggacctgctgttcaagaccaacagaaaggtgactgtcaagcagttgaaagaagattatttcaaaaaaatcgaatgttttgatagcgtggaaatctccggtgtggaggacagatttaatgcttcactgggcacttaccacgacctgctcaaaatcatcaaagacaaagatttcctcgataacgaggaaaacgaggacatcctggaggacattgtgctgacgctgactctgttcgaggatcgcgagatgatcgaggaacgcctgaagacttatgcccatctcttcgacgacaaggtgatgaaacagctcaaacgccggagatacaccggatggggacggctgtcgagaaaactgattaacggaatcagggataaacaatcgggcaaaactatcctggactttctaaagtcagatgggttcgcaaaccggaatttcatgcagctcatccacgacgactccttgactttcaaggaggatattcagaaagcccaagtctccggacagggagactccctgcatgagcacatcgcgaatctggcaggatcgcccgcaattaagaagggcattctccagactgtgaaggtcgtggacgaactggtcaaagtcatgggacgccataagccagagaacatcgtgatcgagatggcgcgggaaaaccagaccactcagaaggggcagaagaacagcagagaaagaatgaaacgcatcgaagaaggcatcaaggaactcggaagccaaatcctcaaggaacatccggtcgagaatactcagctacagaatgagaagctctacctgtactacctgcaaaatggtcgggatatgtacgtcgatcaagaactagacatcaacaggctgtcggattacgatgtggaccacatcgtgccccaatcattcctgaaggacgattcgattgataataaggtgctcactagatcagacaaaaacagaggaaaatcggacaacgtgccgtcagaagaggtggtgaagaagatgaagaattactggcgccaactgctgaacgcaaagctgattacccagagaaaattcgacaatttgaccaaggcagagcgcggaggtctgtcagagcttgacaaggcggggtttatcaagaggcaactggtcgaaacccgccagatcactaaacacgtggcgcagatcctcgactccagaatgaacactaagtacgacgaaaatgataaattgatccgggaagtcaaggtgattaccctgaaatcaaaactggtgtcagattttcgcaaggacttccaattctacaaggtcagggagatcaacaattaccaccatgcccacgatgcctacctgaacgcggtggtgggcactgcgctcatcaagaagtacccaaaactggaatcggagttcgtgtacggagattacaaggtgtacgacgtgcgcaaaatgatcgctaagtcagagcaggaaatcggaaaagccaccgcgaaatacttcttctactcgaacattatgaattttttcaaaactgagattactcttgcgaacggcgagatccgcaaacgccccttgatcgaaactaatggggaaaccggcgaaatcgtctgggataagggcagggatttcgctaccgtcaggaaggtgctgtcaatgccgcaagtgaatatcgtgaaaaaaaccgaagtccaaaccggaggtttcagcaaagagtccatcctgcccaagcgcaactcggataagctcattgctaggaagaaagattgggaccccaagaaatacggcgggttcgactcgccgaccgtggcatactccgtgctggtggtggccaaggtcgaaaaaggaaaatcaaagaagctgaaatccgtgaaggaattgctgggaatcactatcatggagcggtcctcctttgaaaagaatccaattgactttctggaagcaaagggctacaaggaggtgaaaaaggacctgattatcaaacttccaaagtactcactctttgagctggaaaacggtagaaagagaatgctggcgagcgctggagagctgcagaagggaaatgaactggcactgccgtcgaagtacgtgaattttctttatctggcatcacactacgaaaaactcaaaggttcacctgaagataatgagcaaaagcagctatttgtcgaacaacacaagcattacttggacgaaatcatcgaacagatttcggaattttcaaagagagtgatcctggcggatgccaatctggacaaggtgctctcggcgtacaacaagcatcgcgacaagccgatccgcgagcaagcagagaatatcatccacctgttcaccctaacgaacctgggagcgccggcggccttcaaatatttcgacaccactatcgaccggaaacggtacacctcaaccaaggaagtgctcgacgcaaccctgatccatcagtccatcaccggcctctatgaaacccggatcgacctgtcacagctcggcggtgactcgcgggctgacccgaaaaagaagagaaaggtctac

SEQ ID NO: 18 - DNA, 4887 bp, Francisella tularensis
atgaattttaagatcctgcctatcgcaatcgacctgggcgtcaagaatactggcgtgtttagtgctttttatcagaaggggacctcactggagagactggacaataagaacggaaaagtgtatgaactgtccaaggattcttacactctgctgatgaacaataggaccgcacggagacaccagaggcgaggaattgacaggaaacagctggtgaagcgcctgttcaaactgatctggacagagcagctgaacctggaatgggataaggacactcagcaggccatcagcttcctgtttaatcgacggggattctcttttattactgacggctatagtcctgagtacctgaacatcgtgccagaacaggtcaaggcaatcctgatggacattttcgacgattataatggcgaggacgatctggattcctacctgaaactggccacagagcaagagagtaagatcagcgaaatctacaacaagctgatgcagaagatcctggagttcaagctgatgaaactgtgcaccgacatcaaggacgataaagtgagtaccaagacactgaaagagatcacaagctacgagttcgaactgctggccgattatctggctaactacagcgaatccctgaagacccagaaattttcctacacagacaagcagggcaatctgaaagagctgtcttactaccaccatgataagtacaacatccaggagttcctgaagagacacgccaccatcaatgacaggattctggatacactgctgactgacgatctggacatctggaacttcaacttcgagaagttcgatttcgacaagaacgaggaaaaactgcagaatcaggaagataaggaccacattcaggctcatctgcaccatttcgtgtttgcagtcaataagatcaaaagcgagatggcatccggcgggcgccatcgaagccagtacttccaggaaatcaccaacgtgctggacgagaacaatcaccaggaaggctacctgaaaaacttctgtgagaatctgcataacaagaagtacagcaatctgtccgtgaagaatctggtcaacctgattggaaatctgtccaacctggaactgaagcccctgcgcaaatacttcaacgacaagatccacgctaaagcagaccattgggatgagcagaagtttactgaaacctattgccactggattctgggcgagtggcgggtgggggtcaaggatcaggacaagaaagacggcgcaaagtattcttacaaggacctgtgtaacgagctgaagcagaaagtgactaaggccgggctggtggacttcctgctggagctggacccctgccgaaccattccaccttacctggacaacaataacagaaagccacccaaatgtcagagcctgatcctgaatcccaagtttctggataatcagtatcctaactggcagcagtacctgcaggagctgaagaaactgcagtcaatccagaactacctggacagcttcgaaaccgatctgaaggtgctgaaaagctccaaggaccagccttacttcgtcgagtacaagtctagtaaccagcagatcgcttccggccagcgggattacaaggatctggacgcaagaatcctgcagttcatttttgacagggtgaaggcctctgatgagctgctgctgaacgaaatctatttccaggcaaagaaactgaagcagaaagcctcaagcgagctggaaaagctggagtcctctaagaaactggacgaagtgatcgctaactctcagctgagtcagattctgaagtctcagcacacaaatggaatcttcgagcagggcacttttctgcatctggtgtgcaaatactataagcagcgacagagagccagggacagccgcctgtacatcatgcctgaatatcgatacgataagaaactgcacaagtacaacaacaccggccgctttgacgatgacaaccagctgctgacatattgtaatcataagccccggcagaaaagataccagctgctgaacgacctggcaggagtgctgcaggtctctcctaattttctgaaggataaaatcgggtccgatgacgatctgttcatttctaagtggctggtggagcacatccggggctttaagaaggcctgcgaagacagcctgaaaatccagaaggataacaggggactgctgaatcataagatcaacattgcacgcaataccaagggcaaatgcgagaaagaaatcttcaacctgatctgtaagattgaggggagcgaagacaagaaagggaattataagcacggactggcctacgagctgggagtgctgctgttcggagagccaaacgaggccagcaagcccgaatttgataggaaaatcaagaaattcaattcaatctacagctttgcccagatccagcagattgcctttgctgagaggaaggggaatgcaaacacatgcgccgtgtgtagtgcagacaacgcccatcgcatgcagcagatcaaaattactgagccagtcgaagacaataaggataaaatcattctgtcagcaaaggcacagcgactgcctgcaatcccaacccgaattgtggatggagctgtcaagaaaatggctacaattctggcaaagaatatcgtggacgataattggcagaacattaagcaggtcctgagcgcaaaacaccagctgcatatcccaatcattaccgagtccaacgccttcgagtttgaacccgctctggcagacgtgaagggcaaatctctgaaggatagaaggaagaaagccctggagcgaattagtcccgaaaacatcttcaaggataagaacaacagaatcaaggagtttgctaaggggatttccgcctactctggagctaacctgacagatggggacttcgatggagcaaaggaggaactggatcacatcattcctcgcagccataagaaatatggcactctgaacgacgaggctaatctgatttgcgtgacccggggcgataataagaacaaagggaaccggatcttctgtctgagagacctggccgataattacaagctgaaacagtttgagaccacagacgatctggagatcgaaaagaaaattgccgacaccatctgggatgctaataagaaggacttcaagttcggaaactatcggagcttcatcaatctgacacctcaggagcagaaagcattcagacacgccctgtttctggctgatgaaaacccaatcaagcaggcagtgatcagagccattaataaccgcaaccgaaccttcgtgaatggcacacagaggtattttgctgaggtcctggcaaataacatctacctgcgcgccaagaaagaaaatctgaacactgacaagatcagcttcgattactttggaatccctaccattggaaacggccgagggatcgctgagattcggcagctgtatgaaaaggtggacagtgatatccaggcctacgctaaaggcgacaagccacaggcctcttatagtcacctgattgatgctatgctggcattctgcatcgccgctgacgagcatcggaacgatggatctattggcctggaaatcgacaaaaactatagtctgtaccctctggataagaatactggcgaggtgttcaccaaagacatcttttcacagatcaagattaccgacaacgagttcagcgataagaaactggtcagaaagaaagctattgaagggtttaacacacacagacagatgactagggatggaatctatgcagagaattacctgcctatcctgattcataaggagctgaacgaagtgaggaaggggtacacatggaaaaattccgaggaaatcaaaattttcaagggaaagaaatacgacatccagcagctgaataacctggtgtattgtctgaagtttgtggacaaaccaatcagtattgatatccagatttcaaccctggaggaactgagaaacatcctgactaccaataacattgcagccactgccgagtactattacattaatctgaaaacccagaagctgcacgagtattacatcgaaaattacaacacagccctggggtataagaaatacagcaaggagatggagttcctgaggtccctggcttataggtctgagcgcgtgaagatcaaaagtattgacgatgtcaagcaggtcctggacaaggattcaaacttcatcatcggaaagatcacactgcccttcaagaaagagtggcagcgactgtaccgggaatggcagaacacaactatcaaagacgattatgagtttctgaagagcttctttaatgtgaagtccattactaaactgcacaagaaagtccggaaagacttctctctgcccatcagtacaaacgagggcaagtttctggtgaagagaaaaacttgggataataacttcatctaccagattctgaatgactcagatagcagggcagacgggactaaacccttcattcctgcctttgatatcagcaagaacgagattgtggaagccatcattgacagtttcacctcaaaaaacatcttttggctgccaaagaatattgagctgcagaaggtggacaacaagaacatcttcgccattgataccagcaagtggtttgaggtcgaaacaccatccgacctgcgcgatatcggcattgctaccattcagtacaagatcgacaataactcacgccccaaggtgcgagtcaaactggattacgtgatcgacgatgacagcaagattaactatttcatgaatcactcactgctgaagagccggtatcccgacaaagtcctggagatcctgaagcagagcacaatcattgagttcgaaagttcagggtttaacaaaactattaaggagatgctgggaatgaagctggccggcatctacaatgaaacctccaataac

SEQ ID NO: 19 - DNA, 3366 bp, Streptococcus thermophilus
atgagtgacttagttttaggacttgatatcggtataggttctgttggtgtaggtatccttaacaaagtgacaggagaaattatccataaaaactcacgcatcttcccagcagctcaagcagaaaataacctagtacgtagaacgaatcgtcaaggaagacgcttgacacgacgtaaaaaacatcgtatagttcgtttaaatcgtctatttgaggaaagtggattaatcaccgattttacgaagatttcaattaatcttaacccatatcaattacgagttaagggcttgaccgatgaattgtctaatgaagaactgtttatcgctcttaaaaatatggtgaaacaccgtgggattagttacctcgatgatgctagtgatgacggaaattcatcagtaggagactatgcacaaattgttaaggaaaatagtaaacaattagaaactaagacaccgggacagatacagttggaacgctaccaaacatatggtcaattacgtggtgattttactgttgagaaagatggcaaaaaacatcgcttgattaatgtctttccaacatcagcttatcgttcagaagccttaaggatactgcaaactcaacaagaatttaattcacagattacagatgaatttattaatcgttatctcgaaattttaactggaaaacggaaatattatcatggacccggaaatgaaaagtcacggactgattatggtcgttacagaacgaatggagaaactttagacaatatttttggaattctaattgggaaatgtacattttatccagacgagtttagagcagcaaaagcttcctacacggctcaagaattcaatttgctaaatgatttgaacaatctaacagttcctactgaaaccaaaaagttgagcaaagaacagaagaatcaaatcattaattatgtcaaaaatgaaaaggtaatggggccagcgaaactttttaaatatatcgctaaattactttcttgtgatgttgcagatatcaagggacaccgtatcgacaaatcaggtaaggctgagattcatactttcgaagcctatcgaaaaatgaaaacgcttgaaaccttagatattgagcaaatggatagagaaacgcttgataaattagcctatgtcttaacattaaacactgagagggaaggtattcaagaagctttagaacatgaatttgctgatggtagctttagccagaagcaagttgacgaattggttcaattccgcaaagcaaatagttccatttttggaaaaggatggcataatttttctgtcaaactgatgatggagttaattccagaattgtatgagacgtcagaagagcaaatgactatcctgacacgacttggaaaacaaaaaacaacttcgtcttcaaataaaacaaaatatatagatgagaaactattaactgaagaaatctataatcctgttgttgctaagtctgttcgccaggctataaaaatcgtaaatgcggcgattaaagaatacggagactttgacaatattgtcatcgaaatggctcgtgaaacaaatgaagatgatgaaaagaaagctattcaaaagattcaaaaagccaacaaagatgaaaaagatgcagcaatgcttaaggctgctaaccaatataatggaaaggctgaattaccacatagtgttttccacggtcataagcaattagcgactaaaatccgcctttggcatcagcaaggagaacgttgcctttatactggtaagacaatctcaatccatgatttgataaataatcctaatcagtttgaagtagatcatattttacctctttctatcacattcgatgatagccttgcaaataaggttttggtttatgcaactgctaaccaagaaaaaggacaacgaacaccttatcaggctttagatagtatggatgatgcgtggtctttccgtgaattaaaagcttttgtacgtgagtcaaaaacactttcaaacaagaaaaaagaatacctccttacagaagaagatatttcaaagtttgatgttcgaaagaaatttattgaacgaaatcttgtagatacaagatacgcttcaagagttgtcctcaatgcccttcaagaacactttagagctcacaagattgatacaaaagtttccgtggttcgtggccaatttacatctcaattgagacgccattggggaattgagaagactcgtgatacttatcatcaccatgctgtcgatgcattgattattgccgcctcaagtcagttgaatttgtggaaaaaacaaaagaatacccttgtaagttattcagaagaacaactccttgatattgaaacaggtgaacttattagtgatgatgagtacaaggaatctgtgttcaaagccccttatcaacattttgttgatacattgaagagtaaagaatttgaagacagtatcttattctcatatcaagtggattctaagtttaatcgtaaaatatcagatgccactatttatgcgacaagacaggctaaagtgggaaaagataagaaggatgaaacttatgtcttagggaaaatcaaagatatctatactcaggatggttatgatgcctttatgaagatttataagaaggataagtcaaaattcctcatgtatcgtcacgacccacaaacctttgagaaagttatcgagccaattttagagaactatcctaataaggaaatgaatgaaaaagggaaagaagtaccatgtaatcctttcctaaaatataaagaagaacatggctatattcgtaaatatagtaaaaaaggcaatggtcctgaaatcaagagtcttaaatactatgatagtaagcttttaggtaatcctattgatattactccagagaatagtaaaaataaagttgtcttacagtcattaaaaccttggagaacagatgtctatttcaataaaaatactggtaaatatgaaattttaggactgaaatatgctgatttacaatttgaaaagaagacaggaacatataagatttcccaggaaaaatacaatggcattatgaaagaagagggtgtagattctgattcagaattcaagtttacactttataaaaatgatttgttactcgttaaagatacagaaacaaaagaacaacagcttttccgttttctttctcgaactatgcctaatgtgaaatattatgtagagttaaagccttattcaaaagataaatttgagaagaatgagtcacttattgaaattttaggttctgcagataagtcaggacgatgtataaaagggctaggaaaatcaaatatttctatttataaggtaagaacagatgtcctaggaaatcagcatatcatcaaaaatgagggtgataagcctaagctagatttt

SEQ ID NO: 20 - DNA, 3246 bp, Neisseria meningitidis
atggctgccttcaaacctaattcaatcaactacatcctcggcctcgatatcggcatcgcatccgtcggctgggcgatggtagaaattgacgaagaagaaaaccccatccgcctgattgatttgggcgtgcgcgtatttgagcgtgccgaagtaccgaaaacaggcgactcccttgccatggcaaggcgtttggcgcgcagtgttcgccgcctgacccgccgtcgcgcccaccgcctgcttcggacccgccgcctattgaaacgcgaaggcgtattacaagccgccaattttgacgaaaacggcttgattaaatccttaccgaatacaccatggcaacttcgcgcagccgcattagaccgcaaactgacgcctttagagtggtcggcagtcttgttgcatttaatcaaacatcgcggctatttatcgcaacggaaaaacgagggcgaaactgccgataaggagcttggcgctttgcttaaaggcgtagccggcaatgcccatgccttacagacaggcgatttccgcacaccggccgaattggctttaaataaatttgagaaagaaagcggccatatccgcaatcagcgcagcgattattcgcatacgttcagccgcaaagatttacaggcggagctgattttgctgtttgaaaaacaaaaagaatttggcaatccgcatgtttcaggcggccttaaagaaggtattgaaaccctactgatgacgcaacgccctgccctgtccggcgatgccgttcaaaaaatgttggggcattgcaccttcgaaccggcagagccgaaagccgctaaaaacacctacacagccgaacgtttcatctggctgaccaagctgaacaacctgcgtattttagagcaaggcagcgagcggccattgaccgataccgaacgcgccacgcttatggacgagccatacagaaaatccaaactgacttacgcacaagcccgtaagctgctgggtttagaagataccgcctttttcaaaggcttgcgctatggtaaagacaatgccgaagcctcaacattgatggaaatgaaggcctaccatgccatcagccgtgcactggaaaaagaaggattgaaagacaaaaaatccccattaaacctttctcccgaattacaagacgaaatcggcacggcattctccctgttcaaaaccgatgaagacattacaggccgtctgaaagaccgtatacagcccgaaatcttagaagcgctgttgaaacacatcagcttcgataagttcgtccaaatttccttgaaagcattgcgccgaattgtgcctctaatggaacaaggcaaacgttacgatgaagcctgcgccgaaatctacggagaccattacggcaagaagaatacggaagaaaagatttatctgccgccgattcccgccgacgaaatccgcaaccccgtcgtcttgcgcgccttatctcaagcacgtaaggtcattaacggcgtggtacgccgttacggctccccagctcgtatccatattgaaactgcaagggaagtaggtaaatcgtttaaagaccgcaaagaaattgagaaacgccaagaagaaaaccgcaaagaccgggaaaaagccgccgccaaattccgagagtatttccccaattttgtcggagaacccaaatccaaagatattctgaaactgcgcctgtacgagcaacaacacggcaaatgcctgtattcgggcaaagaaatcaacttaggccgtctgaacgaaaaaggctatgtcgaaatcgaccatgccctgccgttctcgcgcacatgggacgacagtttcaacaataaagtactggtattgggcagcgaaaaccaaaacaaaggcaatcaaaccccttacgaatacttcaacggcaaagacaacagccgcgaatggcaggaatttaaagcgcgtgtcgaaaccagccgtttcccgcgcagtaaaaaacaacggattctgctgcaaaaattcgatgaagacggctttaaagaacgcaatctgaacgacacgcgctacgtcaaccgtttcctgtgtcaatttgttgccgaccgtatgcggctgacaggtaaaggcaagaaacgtgtctttgcatccaacggacaaattaccaatctgttgcgcggcttttggggattgcgcaaagtgcgtgcggaaaacgaccgccatcacgccttggacgccgtcgtcgttgcctgctcgaccgttgccatgcagcagaaaattacccgttttgtacgctataaagagatgaacgcgtttgacggtaaaaccatagacaaagaaacaggagaagtgctgcatcaaaaaacacacttcccacaaccttgggaatttttcgcacaagaagtcatgattcgcgtcttcggcaaaccggacggcaaacccgaattcgaagaagccgataccctagaaaaactgcgcacgttgcttgccgaaaaattatcatctcgccccgaagccgtacacgaatacgttacgccactgtttgtttcacgcgcgcccaatcggaagatgagcgggcaagggcatatggagaccgtcaaatccgccaaacgactggacgaaggcgtcagcgtgttgcgcgtaccgctgacacagttaaaactgaaagacttggaaaaaatggtcaatcgggagcgcgaacctaagctatacgaagcactgaaagcacggctggaagcacataaagacgatcctgccaaagcctttgccgagccgttttacaaatacgataaagcaggcaaccgcacccaacaggtaaaagccgtacgcgtagagcaagtacagaaaaccggcgtatgggtgcgcaaccataacggtattgccgacaacgcaaccatggtgcgcgtagatgtgtttgagaaaggcgacaagtattatctggtaccgatttacagttggcaggtagcgaaagggattttgccggatagggctgttgtacaaggaaaagatgaagaagattggcaacttattgatgatagtttcaactttaaattctcattacaccctaatgatttagtcgaggttataacaaaaaaagctagaatgtttggttactttgccagctgccatcgaggcacaggtaatatcaatatacgcattcatgatcttgatcataaaattggcaaaaatggaatactggaaggtatcggcgtcaaaaccgccctttcattccaaaaataccaaattgacgaactgggcaaagaaatcagaccatgccgtctgaaaaaacgcccgcctgtccgt

SEQ ID NO: 21 - DNA, 2952 bp, Campylobacter jejuni
atggcccgcatcctcgctttcgacatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgaccacatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaa

SEQ ID NO: 22 - DNA, 3921 bp, Acidaminococcus sp.
atgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatgagaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggcctctggctgggacgtgaataaggagaagaacaatggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgatcggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaac

SEQ ID NO: 23 - DNA, 3684 bp, Unknown
  Lachnospiraceae
atgtcaaagctcgagaaattcaccaactgttattcgttgagcaaaacactgcggtttaaagcgattccagtcggcaagactcaagagaatatagacaataagcggctgttggtggaagatgaaaagcgcgcggaagactacaaaggggtgaagaagttgttggacagatactacctctcttttatcaatgatgtcttgcactcaatcaaattgaagaatctgaacaactacatctccctcttcagaaagaaaacaaggacagaaaaggagaataaggaacttgaaaatttggagatcaatctgaggaaagagatcgcgaaagcctttaaaggcaacgaaggatacaaaagtctgttcaagaaggatataattgagacaattttgccagagttcctcgatgacaaggacgagattgcgctggtcaattcgttcaacggattcacaacagcattcacaggcttctttgataatcgggaaaatatgttctctgaggaggcaaagtccacttctattgcgttcaggtgtatcaatgagaatctcactaggtacatttccaacatggatatctttgagaaggttgacgcaatttttgacaagcacgaagttcaggagattaaggagaagatcctcaattccgattatgacgttgaggacttcttcgaaggtgagttttttaatttcgtgctcactcaagagggtatcgacgtgtataatgcgatcatcggtgggttcgtgactgagtccggtgaaaagattaagggattgaacgagtatatcaacctttacaaccaaaagacgaaacagaagctgccaaagttcaagcctctttacaaacaggttctttcagaccgcgagtcactctcgttctatggggagggctacacttcggatgaggaagtcctggaggtgttcaggaatactctcaataagaattcggagattttctcttctataaaaaaactggaaaagttgtttaagaattttgacgaatactctagcgccggcatatttgtgaaaaacggcccggccatatcaacgataagtaaagatatcttcggcgaatggaacgtgatcagagacaaatggaacgcggagtatgacgatattcacctgaagaagaaggctgtcgtaacggagaagtacgaggatgatcgcaggaaaagcttcaaaaagatcggaagtttcagcctggaacagttgcaggagtatgctgacgccgatcttagcgtcgtcgagaagttgaaggagataatcatccaaaaggtcgacgagatatataaagtctatggatcaagtgaaaaactgttcgacgccgacttcgttttggagaagtccctgaagaagaacgacgctgttgttgccattatgaaggatctgctcgacagcgtgaagagtttcgagaactatattaaggcttttttcggggaggggaaggagactaacagagatgagtccttctacggagacttcgtcctcgcgtacgatatactccttaaggtagaccacatctacgacgcaatcagaaattacgtgacacaaaagccgtacagcaaggacaagttcaaactctacttccagaacccccagttcatgggcggctgggacaaggacaaggaaacggattacagggctacgatcctgaggtatggttcaaaatactacttggcgattatggacaagaagtacgccaagtgtctccagaagattgacaaagacgatgtcaatggcaattatgagaagatcaactacaagctgcttccgggtccgaacaagatgctcccaaaggttttcttcagcaagaaatggatggcctactataacccaagcgaggacatccagaagatttataagaacggtacgttcaagaagggcgacatgttcaatcttaacgactgtcacaagctgatcgacttcttcaaagactcaattagccggtacccaaagtggtctaacgcctatgacttcaacttttcggaaaccgagaagtacaaggatatagccggattttatagagaggtggaagagcagggctacaaggtgtcattcgagtccgccagcaagaaggaagtggacaagctcgtggaagagggtaagctctacatgttccagatttataataaagactttagcgataagagccacgggacacctaatctccacacaatgtatttcaagctgctcttcgacgagaataaccacggccaaatcaggttgtcaggaggggctgaactcttcatgcggcgcgctagccttaagaaggaggagcttgtagtccaccctgcgaatagtccaattgcgaataagaacccggacaatcctaaaaagactacaacattgagctacgacgtgtacaaggataagaggttttccgaggatcagtacgagctccacatcccgattgcgatcaacaagtgcccaaagaatattttcaagataaacacagaggtgcgtgtactcctgaagcatgacgacaatccttacgtcattgggattgatcggggcgagaggaacctcctctatattgtggtggtggacgggaaggggaacatagtcgaacagtactcccttaacgaaataattaacaatttcaacggcatccgtatcaagaccgactaccattcgttgctggacaagaaggagaaggagagatttgaggcgcggcaaaattggacaagtatcgagaacatcaaggaactcaaagcaggttatatctctcaagttgtgcataagatatgcgagctggttgagaagtatgacgcagtgatcgctcttgaggacctcaactcgggctttaagaattctagagttaaagtggagaagcaggtctatcaaaagttcgagaagatgcttatagataagctcaactacatggtcgataagaaatcgaacccatgtgccaccggcggcgcactcaaaggttaccaaataacaaacaaattcgagtccttcaaatcgatgagtactcagaatgggttcatattttatataccggcgtggcttacgtctaagatcgacccgtcaactggttttgtcaacctgttgaagacgaaatacacgtccattgccgattcgaaaaagttcatatctagttttgatcgtattatgtacgtcccagaggaagatcttttcgagtttgctctcgactacaaaaacttttcgcggaccgatgcggattacattaaaaaatggaaactctattcgtacggcaacagaatcaggatttttcgcaaccctaagaagaataacgtctttgattgggaggaagtttgcttgactagcgcgtacaaggagctctttaataagtatggcattaactaccaacagggtgatatcagagcactgctttgcgaacaatctgacaaggctttctactcatccttcatggctttgatgagcctgatgctccagatgagaaattcaattacaggcagaaccgacgtggatttcttgatctccccggttaaaaattctgatggcatcttttacgatagcaggaactatgaagcgcaagagaatgcgattctgccaaaaaatgcagacgccaacggtgcctataacatcgccaggaaagtcctgtgggcgatcggccagttcaaaaaggccgaagacgaaaaattggacaaggtcaaaatcgctatcagcaacaaagagtggctggagtatgctcagacatccgtaaagcat

SEQ ID NO: 24 - protein, 1068 aa, Artificial Sequence
  Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLANALNDLNNLVITRDENEKLEYYEKFQIIENVFKQKKKPTLKQIAKEILVNEEDIKGYAVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 25 - protein, 1068 aa, Artificial Sequence
  Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLANALNDLNNLVITRDENEKLEYYEKFQIIENVFKQKKKPTLKQIAKEILVNEEDIKGYAVTSTGKPEFTNLKVYHDIKDITARKEIIENAELLDQIAKILTIYQSSEDIQEELTNLNSELTQEEIEQISNLKGYTGTHNLSLKAINLILDELWHTNDNAIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 26 - protein, 1068 aa, Artificial Sequence
  Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLANALNDLNNLVITRDENEKLEYYEKFQIIENVFKQKKKPTLKQIAKEILVNEEDIKGYAVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 27 - protein, 1068 aa, Artificial Sequence
  Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLANALNDLNNLVITRDENEKLEYYEKFQIIENVFKQKKKPTLKQIAKEILVNEEDIKGYAVTSTGKPEFTNLKVYHDIKDITARKEIIENAELLDQIAKILTIYQSSEDIQEELTNLNSELTQEEIEQISNLKGYTGTHNLSLKAINLILDELWHTNDNAIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 28 - protein, 1380 aa, Artificial Sequence
  Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYATVYNELTKVKYVTEGMRKPAFLSGEQKKAIVDLLFKTNRKVTVKQLKEDYFKKIECFDSVAISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVY

SEQ ID NO: 29 - protein, 1380 aa, Artificial Sequence
  Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYATVYNELTKVKYVTEGMRKPAFLSGEQKKAIVDLLFKTNRKVTVKQLKEDYFKKIECFDSVAISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDAFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVY

SEQ ID NO: 30 - protein, 1380 aa, Artificial Sequence
  Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYATVYNELTKVKYVTEGMRKPAFLSGEQKKAIVDLLFKTNRKVTVKQLKEDYFKKIECFDSVAISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITAHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVY

SEQ ID NO: 31 - protein, 1380 aa, Artificial Sequence
  Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYATVYNELTKVKYVTEGMRKPAFLSGEQKKAIVDLLFKTNRKVTVKQLKEDYFKKIECFDSVAISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDAFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITAHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVY

SEQ ID NO: 32 - DNA, 3204 bp, Artificial Sequence
  Staphylococcus aureus
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggcgaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctggccaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacgccgtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggcgaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 33 - DNA, 3204 bp, Artificial Sequence
  Staphylococcus aureus
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggcgaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctggccaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacgccgtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaacgccatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggcgaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 34 - DNA, 3204 bp, Artificial Sequence
  Staphylococcus aureus
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggcgaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctggccaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacgccgtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggcgaagggcaagggcagaatcagcaagaccaagaaagagtatctgctggaagaacgggacatcaacaggttctccgtgcagaaagacttcatcaaccggaacctggtggataccagatacgccaccgccggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 35 - DNA, 3204 bp, Artificial Sequence
  Staphylococcus aureus
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggcgaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctggccaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacgccgtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaacgccatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggcgaagggcaagggcagaatcagcaagaccaagaaagagtatctgctggaagaacgggacatcaacaggttctccgtgcagaaagacttcatcaaccggaacctggtggataccagatacgccaccgccggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 36 - DNA, 4140 bp, Artificial Sequence
  Streptococcus pyogenes
atggataagaaatactcaatcggactggacatcggcactaattcggtgggatgggcagtgattaccgatgagtacaaggtcccgtcgaagaagtttaaggtgctgggaaacaccgatagacactcgatcaagaagaacctcatcggcgctctgctgtttgattccggcgagactgcggaagccacccggctgaagcgcactgcccgcagaagatacacccggcggaaaaatagaatctgctacctccaggagattttctcgaacgaaatggcaaaggtggacgattcattcttccatcgcctggaagagagcttcctggtggaagaggataagaagcatgagcgccatcctatcttcgggaacattgtggacgaagtggcctatcacgagaaatacccgactatctatcatctccgcaagaagctggtggactcaaccgataaggccgacctgaggctgatctacctggcactggcacatatgattaaattccggggccatttccttatcgagggggatctgaatccggataactccgatgtcgataagctgttcatccagctggtccagacttacaatcagctgttcgaggaaaatccgatcaatgcgtcgggcgtggacgccaaggccatcctctcggcgaggctgtcaaaaagcaggcgcctcgaaaacctgatcgcgcagctgccgggggaaaaaaagaacggcctgtttgggaatctgatcgccctctcactgggactaaccccgaatttcaagtcgaatttcgatctcgcagaagacgccaagctgcaattgtccaaggatacctacgacgatgatctggataatctactggctcagatcggagatcagtacgcggacctgttcttggccgctaagaacctctcggacgccattctgctgagcgacatcctgcgcgtcaatactgagatcaccaaagcgccactgagcgcgtcgatgattaagagatacgacgaacatcatcaggatctgactctgctgaaggccctcgtccgccagcagctcccggaaaagtacaaggagattttctttgaccagtcgaagaatggctacgcaggctatatcgacggaggcgccagccaagaagagttttacaagttcatcaagccaatcctggagaagatggacggcactgaggaactgctcgtgaagctgaaccgggaggacttgctgagaaaacagagaaccttcgacaatggaagcatcccgcaccagatccatctcggggaactgcatgctattctgagaaggcaagaggatttctacccctttctgaaggataacagagaaaagatcgaaaagattttgaccttccgcatcccatactacgtggggcctctagccaggggcaacagccggtttgcatggatgacccgcaagtcggaggaaacgatcactccgtggaacttcgaggaagtggtggacaaaggagcgtcagctcagtcctttatcgaacgcatgacgaacttcgataagaatctcccaaacgaaaaggtgcttccaaagcattcgctgctctacgaatacgccaccgtgtacaacgaactcactaaggtgaagtacgtcactgaagggatgcgcaagccggcatttctgtcgggagagcaaaagaaagcaatcgtggacctgctgttcaagaccaacagaaaggtgactgtcaagcagttgaaagaagattatttcaaaaaaatcgaatgttttgatagcgtggccatctccggtgtggaggacagatttaatgcttcactgggcacttaccacgacctgctcaaaatcatcaaagacaaagatttcctcgataacgaggaaaacgaggacatcctggaggacattgtgctgacgctgactctgttcgaggatcgcgagatgatcgaggaacgcctgaagacttatgcccatctcttcgacgacaaggtgatgaaacagctcaaacgccggagatacaccggatggggacggctgtcgagaaaactgattaacggaatcagggataaacaatcgggcaaaactatcctggactttctaaagtcagatgggttcgcaaaccggaatttcatgcagctcatccacgacgactccttgactttcaaggaggatattcagaaagcccaagtctccggacagggagactccctgcatgagcacatcgcgaatctggcaggatcgcccgcaattaagaagggcattctccagactgtgaaggtcgtggacgaactggtcaaagtcatgggacgccataagccagagaacatcgtgatcgagatggcgcgggaaaaccagaccactcagaaggggcagaagaacagcagagaaagaatgaaacgcatcgaagaaggcatcaaggaactcggaagccaaatcctcaaggaacatccggtcgagaatactcagctacagaatgagaagctctacctgtactacctgcaaaatggtcgggatatgtacgtcgatcaagaactagacatcaacaggctgtcggattacgatgtggaccacatcgtgccccaatcattcctgaaggacgattcgattgataataaggtgctcactagatcagacaaaaacagaggaaaatcggacaacgtgccgtcagaagaggtggtgaagaagatgaagaattactggcgccaactgctgaacgcaaagctgattacccagagaaaattcgacaatttgaccaaggcagagcgcggaggtctgtcagagcttgacaaggcggggtttatcaagaggcaactggtcgaaacccgccagatcactaaacacgtggcgcagatcctcgactccagaatgaacactaagtacgacgaaaatgataaattgatccgggaagtcaaggtgattaccctgaaatcaaaactggtgtcagattttcgcaaggacttccaattctacaaggtcagggagatcaacaattaccaccatgcccacgatgcctacctgaacgcggtggtgggcactgcgctcatcaagaagtacccaaaactggaatcggagttcgtgtacggagattacaaggtgtacgacgtgcgcaaaatgatcgctaagtcagagcaggaaatcggaaaagccaccgcgaaatacttcttctactcgaacattatgaattttttcaaaactgagattactcttgcgaacggcgagatccgcaaacgccccttgatcgaaactaatggggaaaccggcgaaatcgtctgggataagggcagggatttcgctaccgtcaggaaggtgctgtcaatgccgcaagtgaatatcgtgaaaaaaaccgaagtccaaaccggaggtttcagcaaagagtccatcctgcccaagcgcaactcggataagctcattgctaggaagaaagattgggaccccaagaaatacggcgggttcgactcgccgaccgtggcatactccgtgctggtggtggccaaggtcgaaaaaggaaaatcaaagaagctgaaatccgtgaaggaattgctgggaatcactatcatggagcggtcctcctttgaaaagaatccaattgactttctggaagcaaagggctacaaggaggtgaaaaaggacctgattatcaaacttccaaagtactcactctttgagctggaaaacggtagaaagagaatgctggcgagcgctggagagctgcagaagggaaatgaactggcactgccgtcgaagtacgtgaattttctttatctggcatcacactacgaaaaactcaaaggttcacctgaagataatgagcaaaagcagctatttgtcgaacaacacaagcattacttggacgaaatcatcgaacagatttcggaattttcaaagagagtgatcctggcggatgccaatctggacaaggtgctctcggcgtacaacaagcatcgcgacaagccgatccgcgagcaagcagagaatatcatccacctgttcaccctaacgaacctgggagcgccggcggccttcaaatatttcgacaccactatcgaccggaaacggtacacctcaaccaaggaagtgctcgacgcaaccctgatccatcagtccatcaccggcctctatgaaacccggatcgacctgtcacagctcggcggtgactcgcgggctgacccgaaaaagaagagaaaggtctac

SEQ ID NO: 37 - DNA, 4140 bp, Artificial Sequence
  Streptococcus pyogenes
atggataagaaatactcaatcggactggacatcggcactaattcggtgggatgggcagtgattaccgatgagtacaaggtcccgtcgaagaagtttaaggtgctgggaaacaccgatagacactcgatcaagaagaacctcatcggcgctctgctgtttgattccggcgagactgcggaagccacccggctgaagcgcactgcccgcagaagatacacccggcggaaaaatagaatctgctacctccaggagattttctcgaacgaaatggcaaaggtggacgattcattcttccatcgcctggaagagagcttcctggtggaagaggataagaagcatgagcgccatcctatcttcgggaacattgtggacgaagtggcctatcacgagaaatacccgactatctatcatctccgcaagaagctggtggactcaaccgataaggccgacctgaggctgatctacctggcactggcacatatgattaaattccggggccatttccttatcgagggggatctgaatccggataactccgatgtcgataagctgttcatccagctggtccagacttacaatcagctgttcgaggaaaatccgatcaatgcgtcgggcgtggacgccaaggccatcctctcggcgaggctgtcaaaaagcaggcgcctcgaaaacctgatcgcgcagctgccgggggaaaaaaagaacggcctgtttgggaatctgatcgccctctcactgggactaaccccgaatttcaagtcgaatttcgatctcgcagaagacgccaagctgcaattgtccaaggatacctacgacgatgatctggataatctactggctcagatcggagatcagtacgcggacctgttcttggccgctaagaacctctcggacgccattctgctgagcgacatcctgcgcgtcaatactgagatcaccaaagcgccactgagcgcgtcgatgattaagagatacgacgaacatcatcaggatctgactctgctgaaggccctcgtccgccagcagctcccggaaaagtacaaggagattttctttgaccagtcgaagaatggctacgcaggctatatcgacggaggcgccagccaagaagagttttacaagttcatcaagccaatcctggagaagatggacggcactgaggaactgctcgtgaagctgaaccgggaggacttgctgagaaaacagagaaccttcgacaatggaagcatcccgcaccagatccatctcggggaactgcatgctattctgagaaggcaagaggatttctacccctttctgaaggataacagagaaaagatcgaaaagattttgaccttccgcatcccatactacgtggggcctctagccaggggcaacagccggtttgcatggatgacccgcaagtcggaggaaacgatcactccgtggaacttcgaggaagtggtggacaaaggagcgtcagctcagtcctttatcgaacgcatgacgaacttcgataagaatctcccaaacgaaaaggtgcttccaaagcattcgctgctctacgaatacgccaccgtgtacaacgaactcactaaggtgaagtacgtcactgaagggatgcgcaagccggcatttctgtcgggagagcaaaagaaagcaatcgtggacctgctgttcaagaccaacagaaaggtgactgtcaagcagttgaaagaagattatttcaaaaaaatcgaatgttttgatagcgtggccatctccggtgtggaggacagatttaatgcttcactgggcacttaccacgacctgctcaaaatcatcaaagacaaagatttcctcgataacgaggaaaacgaggacatcctggaggacattgtgctgacgctgactctgttcgaggatcgcgagatgatcgaggaacgcctgaagacttatgcccatctcttcgacgacaaggtgatgaaacagctcaaacgccggagatacaccggatggggacggctgtcgagaaaactgattaacggaatcagggataaacaatcgggcaaaactatcctggactttctaaagtcagatgccttcgcaaaccggaatttcatgcagctcatccacgacgactccttgactttcaaggaggatattcagaaagcccaagtctccggacagggagactccctgcatgagcacatcgcgaatctggcaggatcgcccgcaattaagaagggcattctccagactgtgaaggtcgtggacgaactggtcaaagtcatgggacgccataagccagagaacatcgtgatcgagatggcgcgggaaaaccagaccactcagaaggggcagaagaacagcagagaaagaatgaaacgcatcgaagaaggcatcaaggaactcggaagccaaatcctcaaggaacatccggtcgagaatactcagctacagaatgagaagctctacctgtactacctgcaaaatggtcgggatatgtacgtcgatcaagaactagacatcaacaggctgtcggattacgatgtggaccacatcgtgccccaatcattcctgaaggacgattcgattgataataaggtgctcactagatcagacaaaaacagaggaaaatcggacaacgtgccgtcagaagaggtggtgaagaagatgaagaattactggcgccaactgctgaacgcaaagctgattacccagagaaaattcgacaatttgaccaaggcagagcgcggaggtctgtcagagcttgacaaggcggggtttatcaagaggcaactggtcgaaacccgccagatcactaaacacgtggcgcagatcctcgactccagaatgaacactaagtacgacgaaaatgataaattgatccgggaagtcaaggtgattaccctgaaatcaaaactggtgtcagattttcgcaaggacttccaattctacaaggtcagggagatcaacaattaccaccatgcccacgatgcctacctgaacgcggtggtgggcactgcgctcatcaagaagtacccaaaactggaatcggagttcgtgtacggagattacaaggtgtacgacgtgcgcaaaatgatcgctaagtcagagcaggaaatcggaaaagccaccgcgaaatacttcttctactcgaacattatgaattttttcaaaactgagattactcttgcgaacggcgagatccgcaaacgccccttgatcgaaactaatggggaaaccggcgaaatcgtctgggataagggcagggatttcgctaccgtcaggaaggtgctgtcaatgccgcaagtgaatatcgtgaaaaaaaccgaagtccaaaccggaggtttcagcaaagagtccatcctgcccaagcgcaactcggataagctcattgctaggaagaaagattgggaccccaagaaatacggcgggttcgactcgccgaccgtggcatactccgtgctggtggtggccaaggtcgaaaaaggaaaatcaaagaagctgaaatccgtgaaggaattgctgggaatcactatcatggagcggtcctcctttgaaaagaatccaattgactttctggaagcaaagggctacaaggaggtgaaaaaggacctgattatcaaacttccaaagtactcactctttgagctggaaaacggtagaaagagaatgctggcgagcgctggagagctgcagaagggaaatgaactggcactgccgtcgaagtacgtgaattttctttatctggcatcacactacgaaaaactcaaaggttcacctgaagataatgagcaaaagcagctatttgtcgaacaacacaagcattacttggacgaaatcatcgaacagatttcggaattttcaaagagagtgatcctggcggatgccaatctggacaaggtgctctcggcgtacaacaagcatcgcgacaagccgatccgcgagcaagcagagaatatcatccacctgttcaccctaacgaacctgggagcgccggcggccttcaaatatttcgacaccactatcgaccggaaacggtacacctcaaccaaggaagtgctcgacgcaaccctgatccatcagtccatcaccggcctctatgaaacccggatcgacctgtcacagctcggcggtgactcgcgggctgacccgaaaaagaagagaaaggtctac

SEQ ID NO: 38 - DNA, 4140 bp, Artificial Sequence
  Streptococcus pyogenes
atggataagaaatactcaatcggactggacatcggcactaattcggtgggatgggcagtgattaccgatgagtacaaggtcccgtcgaagaagtttaaggtgctgggaaacaccgatagacactcgatcaagaagaacctcatcggcgctctgctgtttgattccggcgagactgcggaagccacccggctgaagcgcactgcccgcagaagatacacccggcggaaaaatagaatctgctacctccaggagattttctcgaacgaaatggcaaaggtggacgattcattcttccatcgcctggaagagagcttcctggtggaagaggataagaagcatgagcgccatcctatcttcgggaacattgtggacgaagtggcctatcacgagaaatacccgactatctatcatctccgcaagaagctggtggactcaaccgataaggccgacctgaggctgatctacctggcactggcacatatgattaaattccggggccatttccttatcgagggggatctgaatccggataactccgatgtcgataagctgttcatccagctggtccagacttacaatcagctgttcgaggaaaatccgatcaatgcgtcgggcgtggacgccaaggccatcctctcggcgaggctgtcaaaaagcaggcgcctcgaaaacctgatcgcgcagctgccgggggaaaaaaagaacggcctgtttgggaatctgatcgccctctcactgggactaaccccgaatttcaagtcgaatttcgatctcgcagaagacgccaagctgcaattgtccaaggatacctacgacgatgatctggataatctactggctcagatcggagatcagtacgcggacctgttcttggccgctaagaacctctcggacgccattctgctgagcgacatcctgcgcgtcaatactgagatcaccaaagcgccactgagcgcgtcgatgattaagagatacgacgaacatcatcaggatctgactctgctgaaggccctcgtccgccagcagctcccggaaaagtacaaggagattttctttgaccagtcgaagaatggctacgcaggctatatcgacggaggcgccagccaagaagagttttacaagttcatcaagccaatcctggagaagatggacggcactgaggaactgctcgtgaagctgaaccgggaggacttgctgagaaaacagagaaccttcgacaatggaagcatcccgcaccagatccatctcggggaactgcatgctattctgagaaggcaagaggatttctacccctttctgaaggataacagagaaaagatcgaaaagattttgaccttccgcatcccatactacgtggggcctctagccaggggcaacagccggtttgcatggatgacccgcaagtcggaggaaacgatcactccgtggaacttcgaggaagtggtggacaaaggagcgtcagctcagtcctttatcgaacgcatgacgaacttcgataagaatctcccaaacgaaaaggtgcttccaaagcattcgctgctctacgaatacgccaccgtgtacaacgaactcactaaggtgaagtacgtcactgaagggatgcgcaagccggcatttctgtcgggagagcaaaagaaagcaatcgtggacctgctgttcaagaccaacagaaaggtgactgtcaagcagttgaaagaagattatttcaaaaaaatcgaatgttttgatagcgtggccatctccggtgtggaggacagatttaatgcttcactgggcacttaccacgacctgctcaaaatcatcaaagacaaagatttcctcgataacgaggaaaacgaggacatcctggaggacattgtgctgacgctgactctgttcgaggatcgcgagatgatcgaggaacgcctgaagacttatgcccatctcttcgacgacaaggtgatgaaacagctcaaacgccggagatacaccggatggggacggctgtcgagaaaactgattaacggaatcagggataaacaatcgggcaaaactatcctggactttctaaagtcagatgggttcgcaaaccggaatttcatgcagctcatccacgacgactccttgactttcaaggaggatattcagaaagcccaagtctccggacagggagactccctgcatgagcacatcgcgaatctggcaggatcgcccgcaattaagaagggcattctccagactgtgaaggtcgtggacgaactggtcaaagtcatgggacgccataagccagagaacatcgtgatcgagatggcgcgggaaaaccagaccactcagaaggggcagaagaacagcagagaaagaatgaaacgcatcgaagaaggcatcaaggaactcggaagccaaatcctcaaggaacatccggtcgagaatactcagctacagaatgagaagctctacctgtactacctgcaaaatggtcgggatatgtacgtcgatcaagaactagacatcaacaggctgtcggattacgatgtggaccacatcgtgccccaatcattcctgaaggacgattcgattgataataaggtgctcactagatcagacaaaaacagaggaaaatcggacaacgtgccgtcagaagaggtggtgaagaagatgaagaattactggcgccaactgctgaacgcaaagctgattacccagagaaaattcgacaatttgaccaaggcagagcgcggaggtctgtcagagcttgacaaggcggggtttatcaagaggcaactggtcgaaacccgccagatcactgcccacgtggcgcagatcctcgactccagaatgaacactaagtacgacgaaaatgataaattgatccgggaagtcaaggtgattaccctgaaatcaaaactggtgtcagattttcgcaaggacttccaattctacaaggtcagggagatcaacaattaccaccatgcccacgatgcctacctgaacgcggtggtgggcactgcgctcatcaagaagtacccaaaactggaatcggagttcgtgtacggagattacaaggtgtacgacgtgcgcaaaatgatcgctaagtcagagcaggaaatcggaaaagccaccgcgaaatacttcttctactcgaacattatgaattttttcaaaactgagattactcttgcgaacggcgagatccgcaaacgccccttgatcgaaactaatggggaaaccggcgaaatcgtctgggataagggcagggatttcgctaccgtcaggaaggtgctgtcaatgccgcaagtgaatatcgtgaaaaaaaccgaagtccaaaccggaggtttcagcaaagagtccatcctgcccaagcgcaactcggataagctcattgctaggaagaaagattgggaccccaagaaatacggcgggttcgactcgccgaccgtggcatactccgtgctggtggtggccaaggtcgaaaaaggaaaatcaaagaagctgaaatccgtgaaggaattgctgggaatcactatcatggagcggtcctcctttgaaaagaatccaattgactttctggaagcaaagggctacaaggaggtgaaaaaggacctgattatcaaacttccaaagtactcactctttgagctggaaaacggtagaaagagaatgctggcgagcgctggagagctgcagaagggaaatgaactggcactgccgtcgaagtacgtgaattttctttatctggcatcacactacgaaaaactcaaaggttcacctgaagataatgagcaaaagcagctatttgtcgaacaacacaagcattacttggacgaaatcatcgaacagatttcggaattttcaaagagagtgatcctggcggatgccaatctggacaaggtgctctcggcgtacaacaagcatcgcgacaagccgatccgcgagcaagcagagaatatcatccacctgttcaccctaacgaacctgggagcgccggcggccttcaaatatttcgacaccactatcgaccggaaacggtacacctcaaccaaggaagtgctcgacgcaaccctgatccatcagtccatcaccggcctctatgaaacccggatcgacctgtcacagctcggcggtgactcgcgggctgacccgaaaaagaagagaaaggtctac

SEQ ID NO: 39 - DNA, 4140 bp, Artificial Sequence
  Streptococcus pyogenes
atggataagaaatactcaatcggactggacatcggcactaattcggtgggatgggcagtgattaccgatgagtacaaggtcccgtcgaagaagtttaaggtgctgggaaacaccgatagacactcgatcaagaagaacctcatcggcgctctgctgtttgattccggcgagactgcggaagccacccggctgaagcgcactgcccgcagaagatacacccggcggaaaaatagaatctgctacctccaggagattttctcgaacgaaatggcaaaggtggacgattcattcttccatcgcctggaagagagcttcctggtggaagaggataagaagcatgagcgccatcctatcttcgggaacattgtggacgaagtggcctatcacgagaaatacccgactatctatcatctccgcaagaagctggtggactcaaccgataaggccgacctgaggctgatctacctggcactggcacatatgattaaattccggggccatttccttatcgagggggatctgaatccggataactccgatgtcgataagctgttcatccagctggtccagacttacaatcagctgttcgaggaaaatccgatcaatgcgtcgggcgtggacgccaaggccatcctctcggcgaggctgtcaaaaagcaggcgcctcgaaaacctgatcgcgcagctgccgggggaaaaaaagaacggcctgtttgggaatctgatcgccctctcactgggactaaccccgaatttcaagtcgaatttcgatctcgcagaagacgccaagctgcaattgtccaaggatacctacgacgatgatctggataatctactggctcagatcggagatcagtacgcggacctgttcttggccgctaagaacctctcggacgccattctgctgagcgacatcctgcgcgtcaatactgagatcaccaaagcgccactgagcgcgtcgatgattaagagatacgacgaacatcatcaggatctgactctgctgaaggccctcgtccgccagcagctcccggaaaagtacaaggagattttctttgaccagtcgaagaatggctacgcaggctatatcgacggaggcgccagccaagaagagttttacaagttcatcaagccaatcctggagaagatggacggcactgaggaactgctcgtgaagctgaaccgggaggacttgctgagaaaacagagaaccttcgacaatggaagcatcccgcaccagatccatctcggggaactgcatgctattctgagaaggcaagaggatttctacccctttctgaaggataacagagaaaagatcgaaaagattttgaccttccgcatcccatactacgtggggcctctagccaggggcaacagccggtttgcatggatgacccgcaagtcggaggaaacgatcactccgtggaacttcgaggaagtggtggacaaaggagcgtcagctcagtcctttatcgaacgcatgacgaacttcgataagaatctcccaaacgaaaaggtgcttccaaagcattcgctgctctacgaatacgccaccgtgtacaacgaactcactaaggtgaagtacgtcactgaagggatgcgcaagccggcatttctgtcgggagagcaaaagaaagcaatcgtggacctgctgttcaagaccaacagaaaggtgactgtcaagcagttgaaagaagattatttcaaaaaaatcgaatgttttgatagcgtggccatctccggtgtggaggacagatttaatgcttcactgggcacttaccacgacctgctcaaaatcatcaaagacaaagatttcctcgataacgaggaaaacgaggacatcctggaggacattgtgctgacgctgactctgttcgaggatcgcgagatgatcgaggaacgcctgaagacttatgcccatctcttcgacgacaaggtgatgaaacagctcaaacgccggagatacaccggatggggacggctgtcgagaaaactgattaacggaatcagggataaacaatcgggcaaaactatcctggactttctaaagtcagatgccttcgcaaaccggaatttcatgcagctcatccacgacgactccttgactttcaaggaggatattcagaaagcccaagtctccggacagggagactccctgcatgagcacatcgcgaatctggcaggatcgcccgcaattaagaagggcattctccagactgtgaaggtcgtggacgaactggtcaaagtcatgggacgccataagccagagaacatcgtgatcgagatggcgcgggaaaaccagaccactcagaaggggcagaagaacagcagagaaagaatgaaacgcatcgaagaaggcatcaaggaactcggaagccaaatcctcaaggaacatccggtcgagaatactcagctacagaatgagaagctctacctgtactacctgcaaaatggtcgggatatgtacgtcgatcaagaactagacatcaacaggctgtcggattacgatgtggaccacatcgtgccccaatcattcctgaaggacgattcgattgataataaggtgctcactagatcagacaaaaacagaggaaaatcggacaacgtgccgtcagaagaggtggtgaagaagatgaagaattactggcgccaactgctgaacgcaaagctgattacccagagaaaattcgacaatttgaccaaggcagagcgcggaggtctgtcagagcttgacaaggcggggtttatcaagaggcaactggtcgaaacccgccagatcactgcccacgtggcgcagatcctcgactccagaatgaacactaagtacgacgaaaatgataaattgatccgggaagtcaaggtgattaccctgaaatcaaaactggtgtcagattttcgcaaggacttccaattctacaaggtcagggagatcaacaattaccaccatgcccacgatgcctacctgaacgcggtggtgggcactgcgctcatcaagaagtacccaaaactggaatcggagttcgtgtacggagattacaaggtgtacgacgtgcgcaaaatgatcgctaagtcagagcaggaaatcggaaaagccaccgcgaaatacttcttctactcgaacattatgaattttttcaaaactgagattactcttgcgaacggcgagatccgcaaacgccccttgatcgaaactaatggggaaaccggcgaaatcgtctgggataagggcagggatttcgctaccgtcaggaaggtgctgtcaatgccgcaagtgaatatcgtgaaaaaaaccgaagtccaaaccggaggtttcagcaaagagtccatcctgcccaagcgcaactcggataagctcattgctaggaagaaagattgggaccccaagaaatacggcgggttcgactcgccgaccgtggcatactccgtgctggtggtggccaaggtcgaaaaaggaaaatcaaagaagctgaaatccgtgaaggaattgctgggaatcactatcatggagcggtcctcctttgaaaagaatccaattgactttctggaagcaaagggctacaaggaggtgaaaaaggacctgattatcaaacttccaaagtactcactctttgagctggaaaacggtagaaagagaatgctggcgagcgctggagagctgcagaagggaaatgaactggcactgccgtcgaagtacgtgaattttctttatctggcatcacactacgaaaaactcaaaggttcacctgaagataatgagcaaaagcagctatttgtcgaacaacacaagcattacttggacgaaatcatcgaacagatttcggaattttcaaagagagtgatcctggcggatgccaatctggacaaggtgctctcggcgtacaacaagcatcgcgacaagccgatccgcgagcaagcagagaatatcatccacctgttcaccctaacgaacctgggagcgccggcggccttcaaatatttcgacaccactatcgaccggaaacggtacacctcaaccaaggaagtgctcgacgcaaccctgatccatcagtccatcaccggcctctatgaaacccggatcgacctgtcacagctcggcggtgactcgcgggctgacccgaaaaagaagagaaaggtctac

(19) United States
(12) Patent Application Publication
(10) Pub. No.: US 2025/0270528 A1
(43) Pub. Date: Aug. 28, 2025

(54) CRISPR POLYPEPTIDES

(71) Applicant: Oxford Genetics Limited, Oxford Oxfordshire (GB)

(72) Inventors: Ryan Cawood, Oxford Oxfordshire (GB); Suzanne Snellenberg, Oxford Oxfordshire (GB); Hind Ghezraoui, Oxford Oxfordshire (GB)

(21) Appl. No.: 18/257,417

(22) PCT Filed: Dec. 15, 2021
(86) PCT No.: PCT/GB2021/053309

§ 371 Date: Jun. 14, 2023

(30) Foreign Application Priority Data
Dec. 16, 2020 (GB) 2019908.9

(51) Int. Cl. C12N 9/22; C12N 15/11; C12N 15/90

(52) U.S. Cl. CPC C12N 9/226; C12N 15/11; C12N 15/907; C12N 2310/20

(57) ABSTRACT

The present invention relates to CRISPR polypeptides, including Cas9 and Cas12 5 polypeptides, with reduced off-target activity. The invention also provides compositions comprising such polypeptides, vectors encoding such polypeptides and methods of using such polypeptides.

### CROSS-REFERENCE

This application is a 371 U.S. national phase of PCT/GB2021/053309, filed Dec. 15, 2021, which claims priority from GB 2019908.9, filed Dec. 16, 2020, both which are incorporated by reference in its entirety.

### STATEMENT REGARDING SEQUENCE LISTING

A computer readable form of the Sequence Listing is filed with this application by electronic submission and is incorporated into this application by reference in its entirety. The sequence listing submitted herewith is contained in the text file created Oct. 19, 2023, entitled “23-0881-WO-US_SubSequenceListing_ST25.txt” and 315,392 bytes in size.

### FIELD OF THE INVENTION

The present invention relates to CRISPR polypeptides, including Cas9 and Cas12 polypeptides, with reduced off-target activity. The invention also provides compositions comprising such polypeptides, vectors encoding such polypeptides and methods of using such polypeptides.

### BACKGROUND OF THE INVENTION

The RNA-guided endonucleases of the CRISPR-Cas9 system, including the most widely used Cas9 from Streptococcus pyogenes (SpCas9), are becoming a robust genome-editing tool in model organisms and hold immense promise for therapeutic applications.

Whilst many current Cas9 polypeptides are capable of high-efficiency gene modifications, issues still remain about the off-target activities of Cas9, i.e. the undesirable production of modifications within the genome at sites other than the desired target (see, for example, Zhang et al., “Off-target Effects in CRISPR/Cas9-mediated Genome Engineering”, Molecular Therapy-Nucleic Acids (2015) 4, e264).

With the objective of limiting potential off-target effects without affecting on-target cleavage efficiencies, SpCas9 variants that contains substitutions predicted to weaken the energetics of the target-site cleavage and recognition have previously been developed using directed mutagenesis methods. One of these approaches redesigned the DNA-binding domain of SpCas9 by introducing alanine substitutions at four positions (N497A/R661A/Q696A/Q926A) creating the SpCas9-HF1 (High-fidelity variant 1) that disrupts the non-specific contact between the SpCas9 and phosphate backbone of DNA target sites (Kleinstiver et al., Nature, 2016, 529 (7587): 490-5).

The SpCas9 variant eSpCas9 1.1 (enhanced SpCas9 version 1.1) created by alanine substitutions at three positions (K848A/K1003A/R1060A) neutralizes the positively-charged residues within the non-target strand groove positioned between the HNH, RuvC and PAM domains therefore prevents strand separation and cutting at off-target sites (Slaymaker et al., Science (2016), vol. 351:6268, pp. 84-88). The REC3 domain of SpCas9 have also been re-engineered by targeted mutagenesis at four positions (N692A/M694A/Q695A/H698A) creating a Hyper accurate SpCas9 (HypaCas9) variant that increases Cas9 proofreading and target discrimination (Chen et al. (2017), Nature vol. 550, pp. 407-410).

These three improved versions of SpCas9 have all been shown to maintain robust on-target editing activity relative to wild-type SpCas9. Indeed, SpCas9-HF has been shown to possess comparable activities (greater than 70% of wild-type SpCas9 activities) for 86% (32/37) of the sgRNAs they tested. Chen's study compared the three improved versions (Chen et al. 2017, Nature vol. 550, pp. 407-410): they showed that the HypaCas9 retained high on-target activity (>70% of WT) at 19/24 endogenous gene sites tested, compared to 18/24 for SpCas9-HF1 and 23/24 for eSpCas9(1.1).

There remains a need, however, for further Cas9 polypeptides which display reduced off-target editing activity while maintaining robust on-target editing activity relative to a wild-type SaCas9.

### SUMMARY OF THE INVENTION

The present invention relates to CRISPR polypeptides, including Cas9 and Cas12 polypeptides, with reduced off-target activity. The invention also provides compositions comprising such polypeptides, vectors encoding such polypeptides and methods of using such polypeptides.

It is an object of the invention to provide CRISPR polypeptides (e.g. Cas9, Cas12 or Cas13) with reduced off-target activity, whilst maintaining on-target activity relative to the corresponding wild-type CRISPR polypeptide

It is another object of the invention to provide compositions comprising such polypeptides, vectors encoding such polypeptides and methods of using such polypeptides. The CRISPR polypeptides of the invention may also be used in AAV vectors to deliver genome-editing components in vivo.

### DESCRIPTION OF THE DRAWINGS

FIG. 1 shows details of the reporter assay plasmid which was used to assay the efficacy of the Cas9 mutant polypeptides.

FIG. 2 shows the percentage ON- and OFF-target editing activity of the above Cas9 mutant polypeptides of the invention on a reporter plasmid compared to that of wild-type SaCas9.

FIGS. 3A-3C shows the percentage ON- and OFF-target editing activity of the above Cas9 mutant polypeptides on three different genomic loci compared to that of wild-type SaCas9.

FIG. 4 shows SaCas9 protein expression in indicated SaCas9 mutants as determined by automated WES using specific SaCas9 antibody.

FIG. 5 shows the percentage of edited read by NGS at the EMX1 ON- and OFF-target among total of edited reads by each SaCas9.

FIG. 6 shows an alignment of the Staphylococcus aureus Cas9 and Streptococcus pyogenes Cas9, where the SaCas9 positions 256, 314, 414 and 654 and their equivalents in SpCas9 are shown in bold and underlined.

FIG. 7 shows an alignment of the Staphylococcus aureus Cas9 and Lachnospiraceae Cas12a, where the SaCas9 positions 256, 314, 414 and 654 and their equivalents in Lachnospiraceae Cas12a are shown in bold and underlined.

### DETAILED DESCRIPTION OF THE INVENTION

In one embodiment, the invention provides a CRISPR polypeptide, wherein the amino acid sequence of the CRISPR polypeptide has:
(a) at least 90% amino acid sequence identity with one or more of the reference sequences given in SEQ ID NOs: 1-15; and
(b) wherein for each reference sequence to which the CRISPR polypeptide has at least 90% sequence identity,
   (i) the amino acid in the CRISPR polypeptide at the position which corresponds to position 256 in SEQ ID NO: 1 is different from the amino acid in that reference sequence which corresponds to position 256 in SEQ ID NO: 1,
and
   (ii) the amino acid in the CRISPR polypeptide at the position which corresponds to position 314 in SEQ ID NO: 1 is different from the amino acid in that reference sequence which corresponds to position 314 in SEQ ID NO: 1.

The invention also provides a CRISPR polypeptide, wherein:
(a) the amino acid in the CRISPR polypeptide at the position which corresponds to position Y256 in SEQ ID NO: 1 is an aliphatic amino acid; and
(b) the amino acid in the CRISPR polypeptide at the position which corresponds to position R314 in SEQ ID NO: 1 is an aliphatic amino acid.

In a further embodiment, the invention provides a CRISPR polypeptide, wherein:
(a) the amino acid sequence of the CRISPR polypeptide has at least 90% amino acid sequence identity with one or more of the sequences given in SEQ ID NOs: 1-15; and
(b) the amino acid sequence of the CRISPR polypeptide has two, three or all of (i)-(iv):
   (i) the amino acid in the CRISPR polypeptide at the position which corresponds to position Y256 in SEQ ID NO: 1 is an aliphatic amino acid;
   (ii) the amino acid in the CRISPR polypeptide at the position which corresponds to position R314 in SEQ ID NO: 1 is an aliphatic amino acid;
   (iii) the amino acid in the CRISPR polypeptide at the position which corresponds to position Q414 in SEQ ID NO: 1 is an aliphatic amino acid;
   (iv) the amino acid in the CRISPR polypeptide at the position which corresponds to position R654 in SEQ ID NO: 1 is an aliphatic amino acid.

In yet a further embodiment, the invention provides a Cas9 polypeptide, wherein the amino acid sequence of the polypeptide is given in or comprises any one of SEQ ID NOs: 24-31.

In yet a further embodiment, the invention provides a nucleic acid molecule encoding a CRISPR polypeptide, Cas9 polypeptide, Cas12 polypeptide or Cas13 polypeptide of the invention, preferably a nucleic acid molecule comprising a nucleotide sequence selected from the group consisting of SEQ ID NOs: 32-39.

In yet a further embodiment, the invention provides a vector or plasmid comprising a nucleic acid molecule of the invention.

In yet a further embodiment, the invention provides a host cell comprising (i) a nucleic acid molecule of the invention; or (ii) a vector or plasmid of the invention.

The invention also provides a kit comprising (i) a CRISPR polypeptide, Cas9 polypeptide, Cas12 polypeptide or Cas13 polypeptide of the invention or a vector or plasmid of the invention; and (ii) a guide RNA.

The invention also provides a method of targeting and/or modifying a target DNA molecule, the method comprising contacting (i) a CRISPR polypeptide, Cas9polypeptide, Cas12 polypeptide or Cas13 polypeptide of the invention; and (ii) a guide RNA having a region complementary to the target DNA, with the target DNA molecule.

The CRISPR enzyme is one which is capable of forming a complex with a CRISPR RNA, e.g. a guide RNA (gRNA) or single guide RNA (sgRNA). When complexed with a CRISPR RNA, the CRISPR enzyme is one which is capable of being targeting to a target DNA which has a nucleotide sequence which is complementary to that of the spacer element in the CRISPR RNA.

In some embodiments of the invention, the CRISPR polypeptide is a recombinant polypeptide. In some embodiments of the invention, the CRISPR polypeptide is an isolated polypeptide.

In some embodiments, the CRISPR polypeptide has nuclease, preferably endonuclease, activity. In other embodiments, the CRISPR polypeptide is nuclease-deficient.

In some embodiments, the CRISPR polypeptide is a CRISPR activator (CRISPRa) or a CRISPR inhibitor (CRISPRi) polypeptide.

Preferably, the CRISPR polypeptide is a Cas9, Cas12 or Cas13 polypeptide. The Cas12 polypeptide may be a Cas12a polypeptide.

The amino acid and nucleotide sequences of numerous reference (wt) CRISPR polypeptides are known in the art, as exemplified herein by SEQ ID NOS: 1-23.

| Organism and | Amino | |
|---|---|---|
| CRISPR enzyme | acid | Nucleotide |
| (wild-type | SEQ ID | SEQ ID |
| or variant) | NO | NO |
| Staphylococcus aureus Cas9 | 1 | 16 |
| (wild-type) | | |
| Streptococcus pyogenes Cas9 | 2 | 17 |
| (wild-type) | | |
| Francisella tularensis | 3 | 18 |
| subsp. novicida Cas9 | | |
| (wild-type) | | |
| Streptococcus thermophilus | 4 | 19 |
| Cas9 1122 aa (Q5M542) strain | | |
| (wild-type) | | |
| Neisseria meningitidis | 5 | 20 |
| Cas9 (1082 aa and PDB 6je9) | | |
| (wild-type) | | |
| Campylobacter jejuni | 6 | 21 |
| Cas9 (984 aa PDB 5X2G) | | |
| (wild-type) | | |
| Acidaminococcus | 7 | 22 |
| sp strain BV3L6 Cas12 | | |
| cpf1(1307aa, PDB 5KK5) | | |
| Lachnospiraceae | 8 | 23 |
| Cas12a cpf1 (1228aa, PDB 6KL9 | | |
| (wild-type) | | |
| Staphylococcus aureus Cas9 | 9 | |
| (KKH (E782K/N968K/R1015H) variant recognising | | |
| NNNRRT PAM site) | | |
| Staphylococcus aureus Cas9 | 10 | |
| (cCas9 42 variant | | |
| recognising NNVRRN, NNVACT, | | |
| NNVATG, NNVATT, NNVGCT, NNVGTG, and | | |
| NNVGTT PAM sites) | | |
| Staphylococcus aureus Cas9 | 11 | |
| (cCas9 v17 variant | | |
| recognising NNVRRN, NNVACT, | | |
| NNVATG, NNVATT, NNVGCT, NNVGTG, and | | |
| NNVGTT PAM sites) | | |
| Staphylococcus aureus Cas9 | 12 | |
| (cCas9 v16 variant | | |
| recognising NNVRRN, NNVACT, | | |
| NNVATG, NNVATT, NNVGCT, NNVGTG, and | | |
| NNVGTT PAM sites) | | |
| Staphylococcus aureus Cas9 | 13 | |
| (cCas9 v21 variant | | |
| recognising NNVRRN, NNVACT, | | |
| NNVATG, NNVATT, NNVGCT, NNVGTG, and | | |
| NNVGTT PAM sites) | | |
| Staphylococcus aureus Cas9 | 14 | |
| (N986R variant | | |
| recognising NNGRRT/C PAM sites) | | |
| Staphylococcus aureus Cas9 | 15 | |
| (N986R + R991L variant | | |
| recognising NNGRRT/C PAM sites) | | |

A further 250 Cas9 sequences from different species are disclosed in WO2017/070633 (as SEQ ID NOs: 10-262 in WO2017/070633). Each of these reference sequences is specifically incorporated herein by reference and may be used in place of any of SEQ ID NOs: 1-15.

The CRISPR polypeptide of the invention preferably has (a) at least 90% amino acid sequence identity with one or more of the reference sequences given in SEQ ID NOs: 1-15. Preferably, the CRISPR polypeptide of the invention has (a) at least 90% amino acid sequence identity with one or more of the reference sequences given in SEQ ID NOs: 1-8. More preferably, the CRISPR polypeptide of the invention has (a) at least 90% amino acid sequence identity with one or more of the reference sequences given in SEQ ID NOs: 1-2.

In other embodiments, the level of amino acid sequence identity is at least 91%, 92%, 93%, 94%, 95%, 96%, 97%, 98% or 99% with one or more of the reference sequences given in SEQ ID NOs: 1-15, preferably in SEQ ID NOs: 1-8, and most preferably in SEQ ID NOs: 1-2. In some embodiments, the level of amino acid sequence identity is at least 95% with one or more of the reference sequences given in SEQ ID NOs: 1-15, preferably in SEQ ID NOs: 1-8, and most preferably in SEQ ID NOs: 1-2.

In some embodiments, the CRISPR polypeptide comprises an HNH domain and a RuvC domain. The RuvC domain consists of three separate motifs (RuvC-I-III): RuvC-I is from residues 1-40aa; RuvC-II is from residues 435-480aa; and RuvC-III is from residues 650-774. The HNH domain is from residues 520-628. (The aforementioned numbering refers to the numbering of the SaCas9 polypeptide sequence.)

The HNH nuclease domain of Cas9 functions to cleave the DNA strand complementary to the guide RNA (gRNA). Its active site consists of a ββα-metal fold, and its histidine 840 activates a water molecule to attack the scissile phosphate, which is more electrophilic due to coordination with a magnesium ion, resulting in cleavage of the 3′-5′ phosphate bond.

In some embodiments, the amino acid sequence of the HNH domain of the Cas9 polypeptide of the invention is at least 80%, at least 85%, at least 90%, at least 92%, at least 95%, at least 96%, at least 97%, at least 98%, at least 99%, or 100% identical to the amino acid sequence of the HNH domain of any of SEQ ID NOs: 1-15.

The RuvC domain of Cas9 or Cas12 cleaves the non-target DNA strand. It is encoded by sequentially-disparate sites which interact in the tertiary structure to form the RuvC cleavage domain. The RuvC domain has an RNase H fold structure. The SaCas9 mutation R654, and corresponding mutations in other CRISPR polypeptides, falls within the RuvC-III domain.

In some embodiments, the amino acid sequence of the RuvC domain of the Cas9 or Cas12 polypeptide of the invention is at least 80%, at least 85%, at least 90%, at least 92%, at least 95%, at least 96%, at least 97%, at least 98%, at least 99%, or 100% identical to the amino acid sequence of the RuvC domain of any of SEQ ID NOs: 1-15.

In some embodiments, the CRISPR polypeptide has nuclease (preferably endonuclease) activity. In such embodiments, the CRISPR polypeptide may, for example, be a wild-type Cas9 or Cas12a (Cpf1), or a variant or derivative thereof which has endonuclease activity.

Examples of CRISPR polypeptides which may be used in this regard include SpCas9, FnCas9, St1Cas9, St3Cas9, NmCas9, SaCas9, AsCpf1, LbCpf1, VQR SpCas9, EQR SpCas9, VRER SpCas9, RHA FnCas9 and KKH SaCas9 (see Komor et al., CRISPR-Based Technologies for the Manipulation of Eukaryotic Genomes, Cell (2017), http://dx.doi.org/10.1016/j.cell.2016.10.044).

In other embodiments, the CRISPR enzyme is an endoribonuclease, e.g. C2c2 or Cas13b, or a variant or derivative thereof.

In some embodiments, the CRISPR polypeptide is a nickase. Preferably, the nickase is a CRISPR polypeptide of the invention which additionally comprises a single mutation of D10A/E477/H701/D704/D556/H557 or N580A in SaCas9, or mutations at corresponding positions in other CRISPR polypeptides.

Cas9 is a large, multi-domain protein containing two distinct nuclease domains. Point mutations can be introduced into Cas9 to reduce or abolish nuclease activity, resulting in a dead Cas9 (dCas9) that still retains its ability to bind DNA in a gRNA-programmed manner. Such dCas9 polypeptides, when fused to another polypeptide or polypeptide domain, can target that polypeptide or domain to virtually any DNA sequence simply by co-expression with an appropriate CRISPR RNA. In these embodiments, the CRISPR polypeptide is preferably a catalytically-inactive or nuclease-deficient polypeptide.

In some embodiments, the CRISPR polypeptide is a polypeptide which has no or substantially no endonuclease activity. Lack of nuclease activity may be assessed using a Surveyor assay to detect DNA repair events (Pinera et al. Nature Methods (2013) 10(10):973-976). This CRISPR polypeptide is unable to cleave dsDNA but it retains the ability to target and bind the DNA.

In some embodiments, the CRISPR polypeptide has no detectable nuclease activity. The CRISPR polypeptide may, for example, be one with a diminished nuclease activity or one whose nuclease activity has been inactivated. The CRISPR polypeptide may, for example, have about 0% of the nuclease activity of the non-mutated or wild-type CRISPR polypeptide; less than 3% or less than 5% of the nuclease activity of the non-mutated or wild-type CRISPR polypeptide. The non-mutated or wild-type CRISPR polypeptide may, for example, be SpCas9.

Reducing the level of nuclease activity is possible by introducing mutations into the RuvC and HNH nuclease domains of the SpCas9 and orthologs thereof. For example utilising one or more mutations in a residue selected from the group consisting of D10, E762, H840, N854, N863, or D986; and more preferably introducing one or more of the mutations selected from the group consisting D10A, E762A, H840A, N854A, N863A or D986A. A preferred pair of mutations is D10A with H840A; more preferred is D10A with N863A of SpCas9 and orthologs thereof.

In some embodiments, the CRISPR polypeptide is a dSaCas9 which comprises the mutations D10A/N580A (or a dSpCas9 or other CRISPR polypeptide with mutations in corresponding positions). This dSaCas9 (or a dSpCas9 or other CRISPR polypeptide) may be fused to a transcriptional activation/repression domain to create CRISPRa/CRISPRi, respectively.

In some embodiments, the CRISPR polypeptide is a dCas9 polypeptide. In some other embodiments, the CRISPR polypeptide is a nuclease-deficient Cas12, i.e. a dCas12 polypeptide.

In yet a further embodiment, there is provided a CRISPR polypeptide of the invention (preferably a dCRISPR polypeptide or a nickase) fused to a DNA-modifying domain or a heterologous functional domain or an effector domain. The domain may be fused to the N-terminus or the C-terminus of the CRISPR polypeptide.

The domain may be linked to the CRISPR polypeptide via a linker. The linker does not interfere with the activity of the linked polypeptide.

Examples of suitable DNA-modifying domains include a deaminase, a nuclease, a nickase, a recombinase, a methyl-transferase, a methylase, an acetylase, an acetyl-transferase, a transcriptional activator and a transcriptional repressor domain.

In some embodiments, a CRISPR polypeptide of the invention may be fused to a polypeptide or protein that has an enzymatic activity. In some embodiments, the enzymatic activity modifies a target DNA.

In some embodiments, the enzymatic activity is nuclease activity, methyl-transferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity or reverse transcriptase activity.

In some cases, the enzymatic activity is nuclease activity. In some cases, the nuclease activity introduces a double-strand break in the target DNA. In some cases, the enzymatic activity modifies a target polypeptide associated with the target DNA. In some cases, the enzymatic activity is methyl-transferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity.

In some cases, the target polypeptide is a histone and the enzymatic activity is methyl-transferase activity, demethylase activity, acetyltransferase activity, deacetylase activity, kinase activity, phosphatase activity, ubiquitin ligase activity or deubiquitinating activity.

In some preferred embodiments, the CRISPR polypeptide of the invention is fused to a deaminase and/or a reverse transcriptase.

In some embodiments, the CRISPR polypeptide is a nickase of the invention (preferably a nickase which additionally comprises the mutation D10A or N580A in SaCas9, or mutations at corresponding positions in other CRISPR polypeptides) which is fused to a deaminase and/or a reverse transcriptase.

Such fusion polypeptides may be used to correct mutations in a genome (e.g. in the genome of a human subject) that are associated with disease, or to generate mutations in a genome (e.g. in the human genome) to increase or decrease or prevent expression of a target gene.

In some embodiments, the CRISPR RNA/CRISPR polypeptide complex comprises one or more functional domains which, when juxtaposed to a target nucleic acid (e.g. a target DNA), promote a desired functional activity, e.g. transcriptional activation of an associated gene. In this case, the aim of the complex is to target the functional domain(s) to the desired target nucleic acid. In some embodiments, the complex may act as a programmable transcription regulator.

Upon binding of the CRISPR RNA to the target nucleic acid, the functional domain is placed in a spatial orientation that allows the functional domain to function in its attributed function.

The functional domains may be attached, directly or indirectly, to the CRISPR RNA, or to the CRISPR polypeptide. Preferably, the functional domains are attached, directly or indirectly, to the CRISPR polypeptide. In some embodiments, the one or more functional domains are attached to the Rec1 domain, the Rec2 domain, the HNH domain, or the PI domain of the SpCas9 protein or any ortholog corresponding to these domains.

In certain embodiments, the one or more functional domains are attached to the Rec1 domain at position 553 or 575; the Rec2 domain at any position of 175-306 or replacement thereof; the HNH domain at any position of 715-901 or replacement thereof; or the PI domain at position 1153 of the SpCas9 protein; or any ortholog corresponding to these domains.

The functional domain is generally a heterologous domain, i.e. a domain which is not naturally found in dCas9.

In some embodiments of the invention, at least one of the one or more functional domains have one or more activities selected from the group consisting of methylase activity, demethylase activity, transcription activation activity, transcription repression activity, transcription release factor activity, histone modification activity, RNA cleavage activity, DNA cleavage activity, nucleic acid binding activity and base-conversion activity.

The functional domain may be an effector domain (e.g. a domain which is capable of stimulating transcription of an associated target gene).

The functional domain is preferably a polypeptide or part thereof, e.g. a domain of a protein which has the desired activity. In some preferred embodiments, the functional domain has transcription activation activity, i.e. the functional domain acts as a transcriptional activator. Preferably, one or more of the functional domains is a transcriptional activator which binds to or activates a promoter, thus promoting transcription of the cognate gene.

Examples of transcription factors include heat-shock transcription factors (e.g. HSF1, VP16, VP64, p65 and MyoDI, p300).

Transcriptional repression may be achieved by blocking transcriptional initiation (e.g. by targeting the sgRNA to a promoter) or by blocking transcriptional elongation (e.g. by targeting the sgRNA to an exon). It may also be achieved by fusing a repressor domain to the CRISPR enzyme which induced heterochromatization (e.g. the KRAB domain).

Examples of transcriptional repressor domains include KRAB domain, a SID domain and a SID4X domain.

In a particularly preferred embodiment, the CRISPR polypeptide is a dCas9 fused to a tripartite complex comprising VP64, p65 and Rta.

Numerous sequence comparison tools are available which can be used to align two amino acid sequences in order to determine the level of amino acid sequence identity and to identify corresponding amino acids in other sequences.

One such alignment program is the NCBI Constraint-based Multiple Alignment Tool (COBALT, accessible at st-va.ncbi.nlm.nih.gov/tools/cobalt), using the following parameters—Alignment parameters: Gap penalties −11, −1; End-Gap penalties −5, −1. CDD Parameters: Use RPS BLAST on; Blast E-value 0.003; Find Conserved columns and Recompute on. Query Clustering Parameters: Use query clusters on; Word Size 4; Max cluster distance 0.8; Alphabet Regular.

Preferably, the alignment program is EMBOSS Needle. This creates an optimal global alignment of two sequences using the Needleman-Wunsch algorithm (https://www.ebi.ac.uk/Tools/psa/emboss_needle/). The preferred parameters are: Output format=pair; Matrix=BLOSUM62; Gap Open=10; Gap extend=0.5; End Gap Penalty=false; End Gap Open=10; End Gap Extend=0.5.

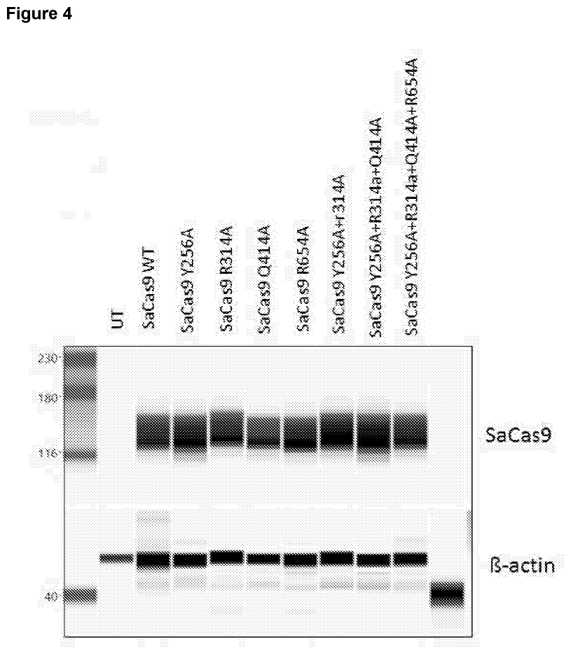

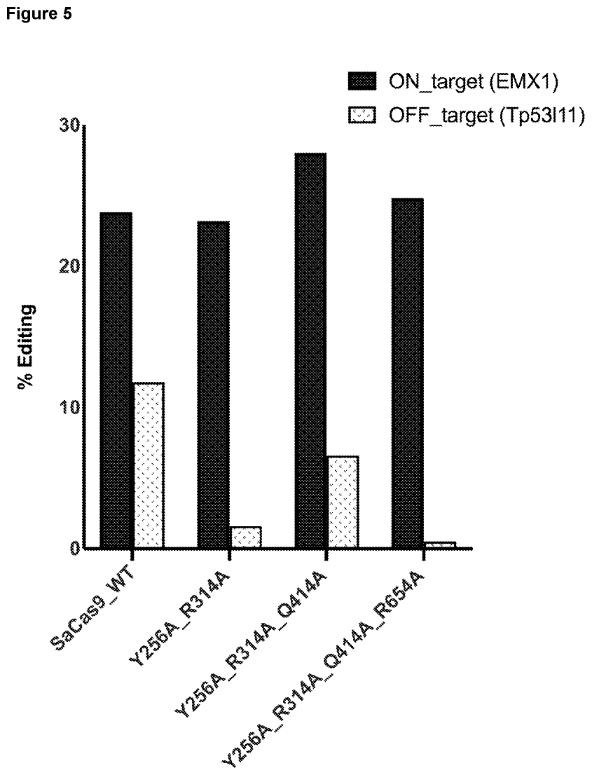

An exemplary alignment of the Staphylococcus aureus Cas9 and Streptococcus pyogenes Cas9 amino acid sequences is shown herein in FIG. 4, where the SaCas9 positions 256, 314, 414 and 654, and their equivalents are shown in bold. A further exemplary alignment of the Staphylococcus aureus Cas9 and Lachnospiraceae Cas12 amino acid sequences is shown herein in FIG. 5, where the SaCas9 positions 256, 314, 414 and 654, and their equivalents are shown in bold.

Other examples of alignments of Cas9 polypeptides are given in WO2017/070633.

These alignments demonstrate that amino acid sequences and specific amino acids that are homologous to a reference Cas9 amino acid sequence or specific amino acid can be identified across Cas9 and Cas12 sequence variants, including, but not limited to Cas9 and Cas12 sequences from different species, by identifying the amino acid sequence or specific amino acid that aligns with the reference sequence or the reference amino using alignment programs and algorithms known in the art.

The following table identifies the specific amino acid positions which correspond to positions Y256, R314, Q414 and R654 in S. aureus:

| | SEQ ID | | | | |
|---|---|---|---|---|---|
| CRISPR polypeptide | NO: (wt) | Mutation position | | | |
| S. aureus | 1 | Y256 | R314 | Q414 | R654 |
| S. pyogenes | 2 | F518 | E579 | G687 | K929 |
| Francisella tularensis | 3 | H639 | K707 | K840 | H1062 |
| subsp. novicida | | | | | |
| Streptococcus thermophilus | 4 | F278 | R338 | Q446 | R692 |
| 1122 aa (Q5M542) strain | | | | | |
| Neisseria meningitidis | 5 | F277 | K333 | K455 | R675 |
| (1082 aa and PDB 6je9) | | | | | |
| Campylobacter jejuni | 6 | F265 | K325 | D415 | R645 |
| Cas9 (984 aa, PDB 5X2G) | | | | | |
| Acidaminococcus | 7 | F138 | R192 | T291 | E588 |
| sp strain BV3L6 Cas12 | | | | | |
| cpf1(1307aa, PDB 5KK5) | | | | | |
| Lachnospiraceae | 8 | Y237 | G291 | D407 | H714 |
| Cas12a Cpf1 | | | | | |
| (1228aa, PDB 6KL9) | | | | | |

For example, it can be seen from the above table that amino acid F265 in Campylobacter jejuni Cas9 corresponds to SaCas9 position Y256.

In some embodiments of the invention, the amino acid in the CRISPR polypeptide which corresponds to one or more of positions Y256, R314, Q414 or R654 in SEQ ID NO: 1 is substituted to become an aliphatic amino acid. As used herein, the term “aliphatic amino acid” refers to an amino acid which is independently selected from the group consisting of alanine, leucine, isoleucine and valine. Each of the aliphatic amino acids (i.e. those corresponding to amino acids at positions Y256, R314, Q414 and R654 of SEQ ID NO: 1) may be the same or different. Preferably, the aliphatic amino acid is alanine.

Another drawback of current CRISPR genome-engineering tools is that they are limited with respect to the DNA sequences that can be targeted. While Cas9 can be targeted to virtually any target sequence by providing a suitable guide RNA, Cas9 technology is still limited with respect to the sequences that can be targeted by a strict requirement for a PAM motif that must be present immediately adjacent to the 3′-end of the targeted DNA sequence in order for the Cas9 protein to bind and act upon the target sequence. The PAM requirement thus limits the sequences that can be efficiently targeted by Cas9 proteins.

The wild-type SaCas9 requires a PAM motif having the nucleotide sequence NNGRRT.

In Cas9 polypeptides having all of the following substitutions in SaCas9 (SEQ ID NO: 1) (or substitutions at corresponding amino acid positions), the PAM site is preferably modified to be NNNRRT: E782K/N968K/R1015H (Kleinstiver et al., Nat. Biotechnol. 2015 December; 33(12): 1293-1298).

In a further embodiment, therefore, the invention provides a CRISPR polypeptide wherein, additionally:
(i) the amino acid in the CRISPR polypeptide at the position which corresponds to position E782 in SEQ ID NO: 1 is lysine (K);
(ii) the amino acid in the CRISPR polypeptide at the position which corresponds to position N968 in SEQ ID NO: 1 is lysine (K); and
(iii) the amino acid in the CRISPR polypeptide at the position which corresponds to position R1015 in SEQ ID NO: 1 is histidine (H).

Ma et al. (Nature Communications, volume 10, Article number: 560 (2019)) have engineered SaCas9 variants with altered PAM recognition specificity. By swapping a key region in the PI domain in SaCas9, they identified several cCas9 v42 and v17-L variants with expanded DNA cleavage activities at NNVRRN PAMs, along with multiple cCas9 v16 and v21 derived variants that can efficiently target sites with NNVACT, NNVATG, NNVATT, NNVGCT, NNVGTG, and NNVGTT PAM.

The following sequences are therefore included herein as Cas9 reference sequences: cCas9 v42 (SEQ ID NO: 10); cCas9 v17 (SEQ ID NO: 11); cCas9 v16 (SEQ ID NO: 12); and cCas9 v21 (SEQ ID NO: 13).

SaCas9 variants bearing the N986R mutation, with or without an additional R991L mutation, enhanced its PAM targeting range (Luan et al., J. Am. Chem. Soc. 2019, 141, 16, 6545-6552 March 29, 2019).

In a further embodiment, therefore, the invention provides a CRISPR polypeptide wherein, additionally:
(i) the amino acid in the CRISPR polypeptide at the position which corresponds to position N986 in SEQ ID NO: 1 is arginine (R); and/or
(ii) the amino acid in the CRISPR polypeptide at the position which corresponds to position R991 in SEQ ID NO: 1 is leucine (L).

In some particularly preferred embodiments, the CRISPR polypeptide of the invention is one having the mutations or corresponding mutations as shown in the following table:

| Parent organism | | SEQ ID NO: | |
|---|---|---|---|
| and polypeptide | Mutations | Amino acid | Nucleotide |
| Staphylococcus | Y256A/R314A | 24 | 32 |
| aureus | Y256A/R314A/Q414A | 25 | 33 |
| Cas9 | Y256A/R314A/R654A | 26 | 34 |
| | Y256A/R314A/ | 27 | 35 |
| | Q414A/R654A | | |
| Streptococcus | F518A/E579A | 28 | 36 |
| pyogenes | F518A/E579A/G687A | 29 | 37 |
| Cas9 | F518A/E579A/K929A | 30 | 38 |
| | F518A/E579A/ | 31 | 39 |
| | G687A/K929A | | |

In a further embodiment, the invention provides a CRISPR polypeptide, wherein:
(a) the amino acid sequence of the CRISPR polypeptide has at least 90% amino acid sequence identity with one or more of the reference sequences given in SEQ ID NOs: 1-15; and
(b) the amino acid sequence of the CRISPR polypeptide has two, three or all of (i)-(iv):
   (i) the amino acid in the CRISPR polypeptide at the position which corresponds to position Y256 in SEQ ID NO: 1 is an aliphatic amino acid;
   (ii) the amino acid in the CRISPR polypeptide at the position which corresponds to position R314 in SEQ ID NO: 1 is an aliphatic amino acid;
   (iii) the amino acid in the CRISPR polypeptide at the position which corresponds to position Q414 in SEQ ID NO: 1 is an aliphatic amino acid;
   (iv) the amino acid in the CRISPR polypeptide at the position which corresponds to position R654 in SEQ ID NO: 1 is an aliphatic amino acid.
      Preferably, the aliphatic amino acid is alanine.

Also provided is a pharmaceutical composition comprising a CRISPR polypeptide or fusion polypeptide of the invention, optionally together with one or more carriers, excipients or diluents. The composition may, for example, comprise one or more of a buffer (e.g. HEPES pH 7.5), sodium chloride, sucrose and dithiothreitol. The composition may be provided in liquid (i.e. aqueous) or in lyophilised form.

In yet a further embodiment, there is provided a complex comprising (i) a CRISPR polypeptide or fusion polypeptide of the invention; and (ii) a guide RNA.

The invention also provides kits comprising (i) a CRISPR polypeptide or fusion polypeptide of the invention; and (ii) a guide RNA.

The guide RNA may be a single guide RNA, a tracrRNA or a crRNA. The guide RNA is preferably 15-100 nucleotides long, more preferably 15-50 or 15-30 nucleotides long. The guide RNA preferably comprises a region of contiguous nucleotides that is complementary to a target sequence, preferably wherein the target sequence is a sequence in the genome of a human.

The on- and off-target activities of the CRISPR polypeptides of the invention may be assayed by any suitable means. For example, a reporter plasmid may be used to evaluate the ability of a Cas9/gRNA complex to bind to a target sequence upstream of a reporter gene or to produce mutations in the target sequence which disrupt the reading frame of the reporter gene. Off-target effects may be analysed using similar reporter plasmid wherein the target sequence is replaced by a non-target sequence. Preferably, the CRISPR polypeptide of the invention has a reduced (e.g. at least 10%, 20%, 30%, 40% or 50% lower) off-target activity compared to wild-type S. aureus Cas9, e.g. in an off-target assay as described in Example 1.

In a further embodiment, there is provided a nucleic acid molecule which encodes a CRISPR polypeptide or a fusion polypeptide of the invention. The nucleic acid molecule may be DNA or RNA. It may be single or double-stranded.

The nucleic acid molecule which encodes a CRISPR polypeptide of the invention preferably includes, but is not limited to:
(a) a polynucleotide molecule whose nucleotide sequence comprises or consists of the nucleotide sequence given in any one of SEQ ID NOs: 32-39;
(b) a polynucleotide molecule whose nucleotide sequence comprises or consists of a variant nucleotide sequence having at least 90%, 95% or 99% sequence identity to any one of SEQ ID NOs: 32-39, the variant nucleotide sequence encoding one or more of the mutations of the invention; and
(c) a polynucleotide molecule whose nucleotide sequence comprises or consists of a nucleotide sequence which encodes:
   (i) a polypeptide whose amino acid sequence is given in any one of SEQ ID NOS: 24-31 or
   (ii) a variant of (i), the variant having at least 90%, 95% or 99% sequence identity to any one of SEQ ID NOs: 24-31, the variant encoding one or more of the mutations of the invention.

In yet a further embodiment, the invention provides a vector or plasmid comprising a nucleic acid molecule of the invention.

In yet another embodiment, the invention provides a viral vector or recombinant virus particle comprising a nucleic acid molecule of the invention. Preferably, the viral vector or recombinant virus particle comprises a transgene, wherein the transgene comprises a nucleic acid molecule of the invention operably-associated with one or more regulatory elements, preferably selected from the group consisting of an enhancer, a promoter and a terminator.

In some embodiments, the viral vector or recombinant virus particle is an adenoviral vector or recombinant adenovirus particle. Preferably, the nucleic acid molecule which encodes a CRISPR polypeptide of the invention or transgene is inserted within one of the adenoviral early region genes. More preferably, the nucleic acid molecule or transgene is inserted within the adenoviral E1 region genes. Even more preferably, the E1A and E1B genes are deleted from the Adenovirus genome and the nucleic acid molecule or transgene is inserted into this region.

In other embodiments, the viral vector or recombinant virus particle is an adeno-associated virus (AAV) vector or recombinant AAV particle. In particular, the invention provides a recombinant AAV genome comprising a nucleic acid molecule which encodes a CRISPR polypeptide of the invention or the transgene.

In some particularly-preferred embodiments, the CRISPR polypeptide is a Cas9 from Staphylococcus aureus, Streptococcus thermophilus or Neisseria meningitidis, having one or more substitutions or mutations of the invention. The genes encoding the latter Cas9 polypeptides are all about 1 kb shorter than the corresponding SpCas9 gene, and hence the latter genes are more readily packaged into AAV vectors.

In some embodiments, the nucleic acid molecule in the recombinant AAV genome encodes a CRISPR polypeptide which additionally comprises a transcriptional repression (CRISPRi) or activator (CRISPRa) domain.

As used herein, the term “recombinant AAV genome” refers to an AAV genome comprising AAV inverted terminal repeats (ITRs) flanking the nucleic acid molecule which encodes a CRISPR polypeptide of the invention or the transgene. As used herein, the term “recombinant AAV particle” refers to an AAV particle which comprises a recombinant AAV genome.

In yet another embodiment, there is provided a cell comprising a CRISPR polypeptide of the invention, a fusion polypeptide of the invention, a nucleic acid molecule of the invention, a vector of the invention or a CRISPR polypeptide: RNA complex of the invention. The cell may be an isolated cell, a host cell and/or a recombinant cell. Also provided is a cell line and a population of cells comprising cells of the invention.

The cell may be one which is used to produce the CRISPR polypeptide of the invention or a fusion polypeptide of the invention. Suitable cells include Escherichia coli cells.

The cell may also be one in which the CRISPR polypeptide of the invention is used to modify the cellular genome. Examples of such cells include, but are not limited to, neurons (preferably retinal neurons), hepatocytes, muscle cells, stem cells (e.g. haematopoietic stem cells, mesenchymal stem cells, embryonic stem cells, adipose stem cells, induced pluripotent stem cells and their derivatives), immune cells (including B and T lymphocytes, natural killer cells, monocytes and macrophages and granulocytes), endothelial cells, cardiovascular cells, epithelial cells, mesenchymal cells, pancreatic b cells or pancreatic a cells, cardiomyocytes, spleen cells, fat cells, glial cells, fibroblasts, Kupffer cells and cancer cells (e.g. leukaemia, lymphoma, myeloma, carcinoma, sarcoma, melanoma cells).

The DNA molecules, plasmids and vectors of the invention may be made by any suitable technique. Recombinant methods for the production of the nucleic acid molecules and packaging cells of the invention are well known in the art (e.g. “Molecular Cloning: A Laboratory Manual” (Fourth Edition), Green, MR and Sambrook, J., (updated 2014)).

The invention also provides methods of using the CRISPR polypeptide or fusion polypeptide of the invention to target a DNA molecule and/or to modify a DNA molecule.

In particular, there is provided a method of targeting and/or modifying a target DNA molecule, the method comprising contacting:
(i) a CRISPR polypeptide or fusion polypeptide of the invention; and
(ii) a guide RNA having a region complementary to the target DNA,
   with the target DNA molecule.

In some embodiments, the target DNA molecule is present in the genome of a cell, preferably a mammalian cell, more preferably a human cell. As used herein, the term “genome” includes the cell's nuclear genome, mitochondrial genomes and plastid genomes. Preferably, the term “genome” relates to the cell's nuclear genome.

The disclosure of each reference set forth herein is specifically incorporated herein by reference in its entirety.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Production and Assay for Cas9 Mutants

SaCas9 mutants were generated using Q5 Site-Directed Mutagenesis Kit along with mutagenic primers to introduce the desired change. We assessed the effect of these mutations on changes to genome-editing specificity. The following SaCas9 mutants were produced:

Y256A/R314A
Y256A/R314A/Q414A
Y256A/R314A/Q414A/R654A

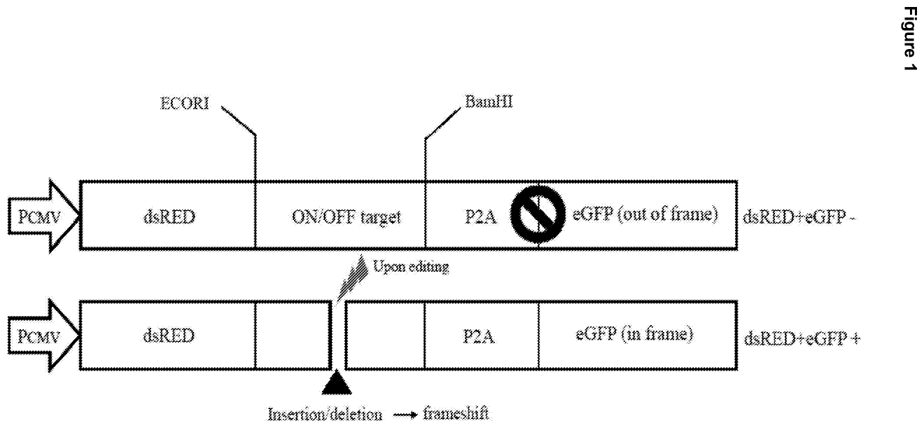

A reporter assay plasmid assay was used to evaluate SaCas9 cleavage efficiency on eGFP expression. This reporter plasmid contained dsRed-Express2 and eGFP expression cassettes, separated by ON/OFF target sites (including PAM) and the self-cleaving peptide P2A (FIG. 1). The EcoRI and BamHI sites were used to clone in the ON/OFF target site DNA. The insertion of the target site DNA disrupted the reading frame of P2A and eGFP, resulting in only dsRed expression. Upon successful editing of the target sites by SaCas9 mutants, INDELs arose, resulting in both dsRed and eGFP expression.

HEK293T cells were co-transfected with plasmids encoding WT SaCas9 or the generated SaCas9 mutants and a guide RNA that targeted the EMX1 gene, and with the reporter plasmids containing either the ON or the OFF target sequence.

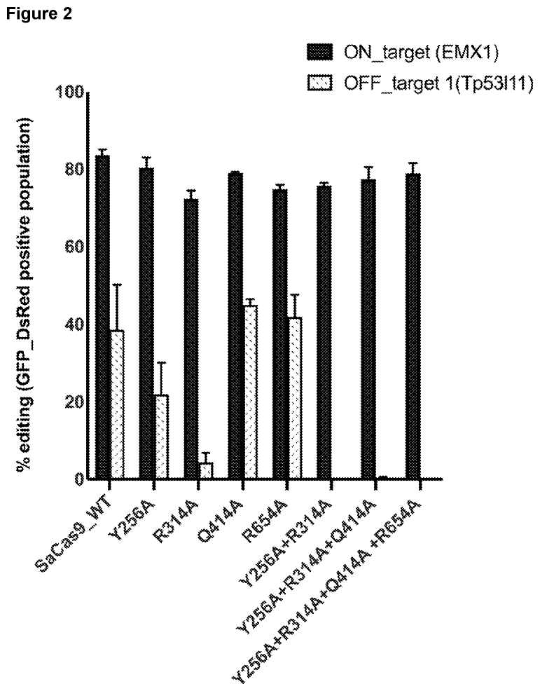

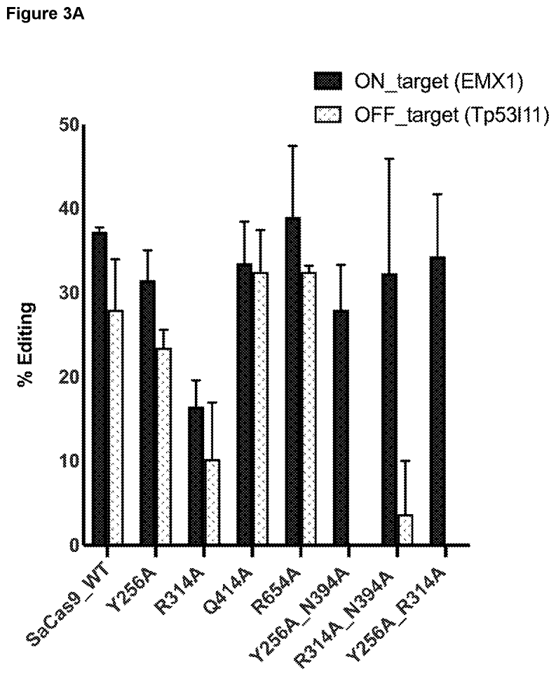

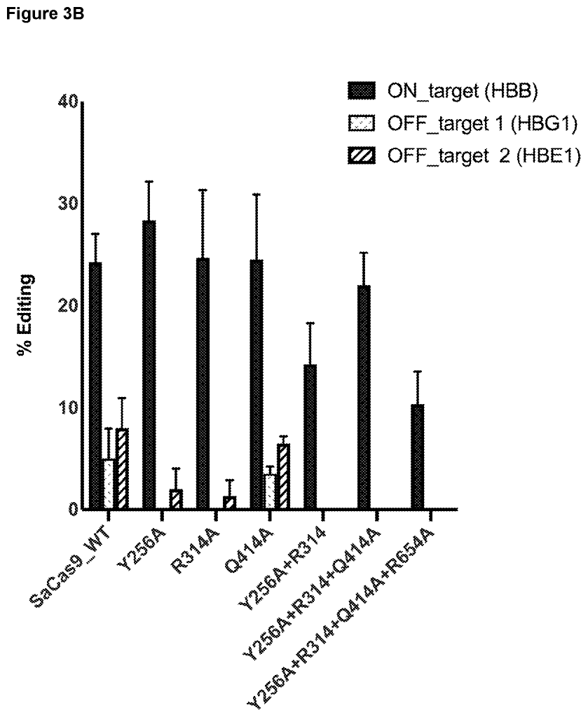

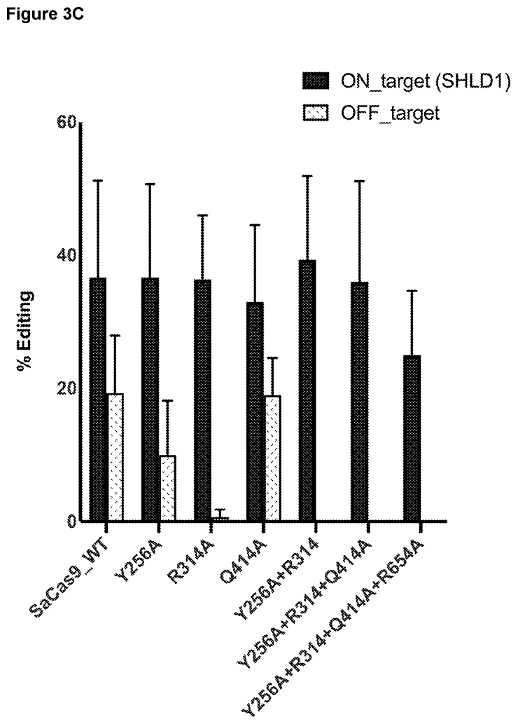

DsRed and eGFP expression was examined by flow cytometry. The selected hits were validated on a genomic level. PCR was performed around the ON target EMX1 locus and around the OFF target site TP53I11, and the PCR products were Sanger sequenced. The results are shown in FIGS. 2 and 3A-C.

FIG. 2 shows the percentage ON- and OFF-target editing activity of the above Cas9 mutant polypeptides of the invention on the reporter plasmid compared to that of wild-type SaCas9. This shows that the double, triple and quadruple mutants all have significantly lower off-target editing than the wild-type Cas9 or the single mutant.

FIGS. 3A-3C shows the percentage ON- and OFF-target editing activity of the above Cas9 mutant polypeptides on three different genomic loci compared to that of wild-type SaCas9. This shows that the double, triple and quadruple mutants all have significantly lower off-target editing than the wild-type Cas9 or the single mutant.

FIG. 4 shows SaCas9 protein expression in indicated SaCas9 mutants as determined by automated WES using specific SaCas9 antibody. All SaCas9 variants showed similar protein levels compared to SaCas9 WT.

FIG. 5 shows the percentage of edited read by NGS at the EMX1 ON- and OFF-target among total of edited reads by each SaCas9. This shows that the double, triple and quadruple mutants all have significantly lower off-target editing than the wild-type Cas9.

### Example 2: Identification of Corresponding Mutants in Other Species

An amino acid sequence alignment was produced between the Staphylococcus aureus Cas9 sequence and the Streptococcus pyogenes Cas9 sequence using the following parameters: Alignment program=EMBOSS Needle; Output format=pair; Matrix=BLOSUM62; Gap Open=10; Gap extend=0.5; End Gap Penalty=false; End Gap Open=10; End Gap Extend=0.5.

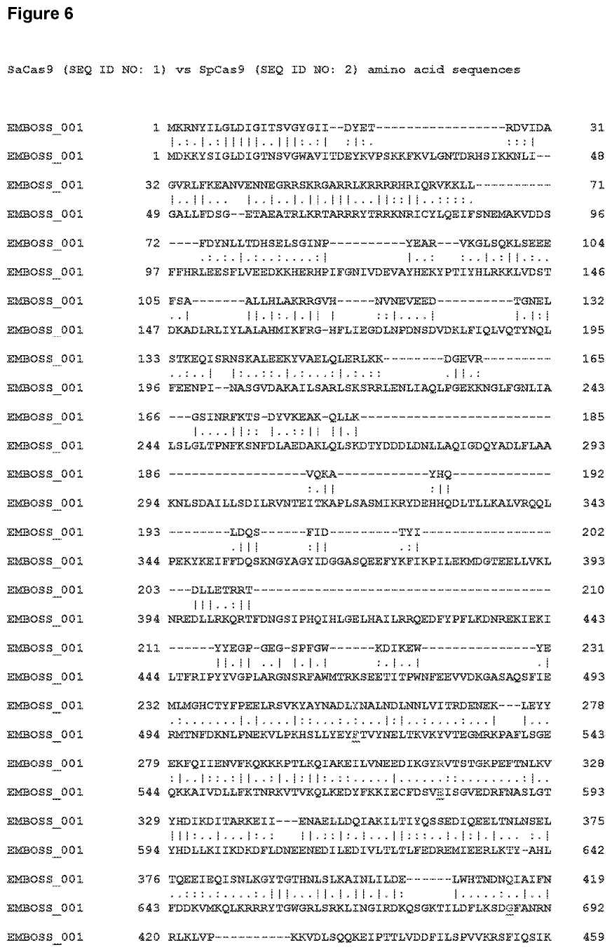

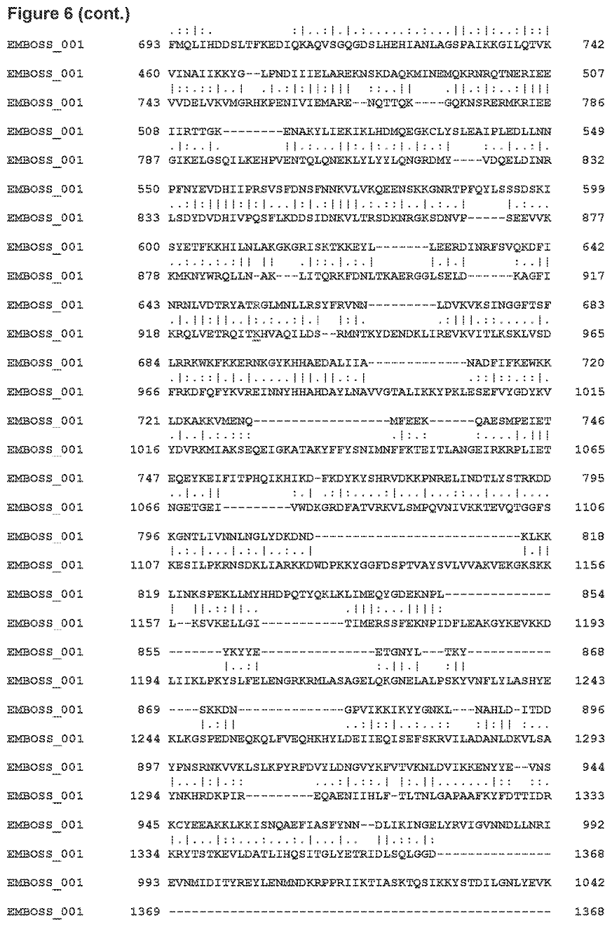

FIG. 6 shows an alignment of the two sequences, where the SaCas9 positions 256, 314, 414 and 654 and their equivalents in SpCas9 are shown in bold and underlined.

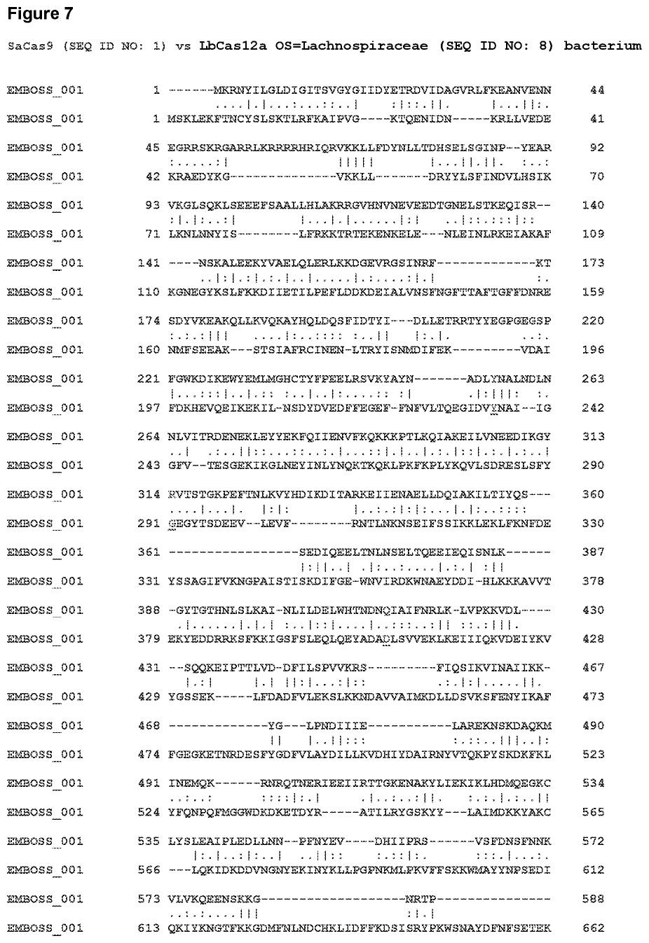

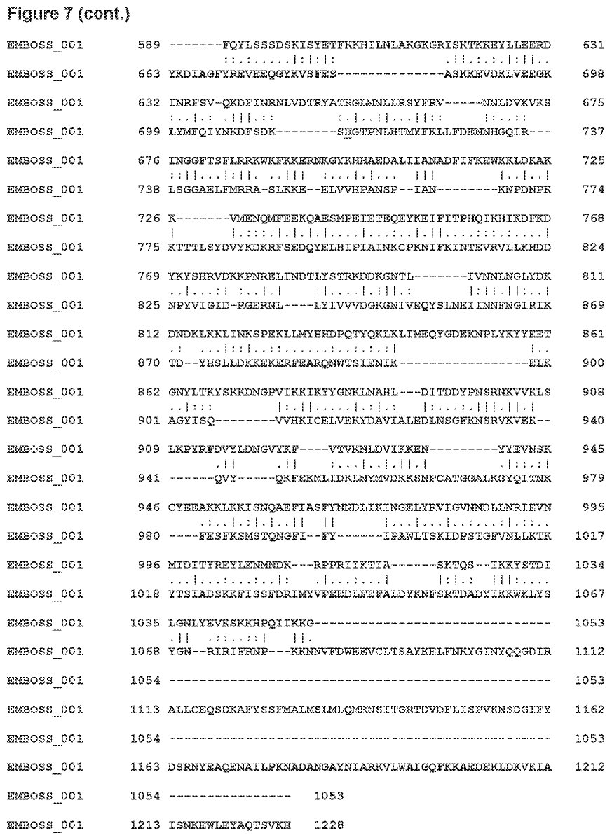

FIG. 7 shows an alignment of the Staphylococcus aureus Cas9 and Lachnospiraceae Cas12a, where the SaCas9 positions 256, 314, 414 and 654 and their equivalents in Lachnospiraceae Cas12a are shown in bold and underlined.

This demonstrates that the amino acids which correspond to positions 256, 314, 414 and 654 in the Staphylococcus aureus Cas9 sequence may readily be identified in other species.

DETAILS OF SEQUENCES
SEQ ID NO: 1
SaCAS9_Protein sequence
SEQ ID NO: 2
SpCas9_protein sequence
SEQ ID NO: 3
>sp|A0Q5Y3|CAS9_FRATN CRISPR-associated endonuclease Cas9
OS = Francisella tularensis subsp. novicida (strain U112) OX = 401614
GN = CAS9_PE = 1 SV = 1
SEQ ID NO: 4
>tr|Q5M542|Q5M542_STRT2 CRISPR-associated endonuclease Cas9
OS = Streptococcus thermophilus (strain ATCC BAA-250/LMG 18311)
OX = 264199 GN = CAS9_PE = 3 SV = 1
SEQ ID NO: 5
>sp|C9X1G5|CAS9_NEIM8 CRISPR-associated endonuclease CAS9_OS = Neisseria
meningitidis serogroup C (strain 8013) OX = 604162 GN = CAS9_PE = 1 SV = 1
SEQ ID NO: 6
>sp|Q0P897|CAS9_CAMJE CRISPR-associated endonuclease Cas9
OS = Campylobacter jejuni subsp. jejuni serotype 0:2 (strain ATCC 700819
/NCTC 11168) OX = 192222 GN = CAS9_PE = 1 SV = 1
SEQ ID NO: 7
sp|U2UMQ6|CS12A ACISB CRISPR-associated endonuclease Cas12a
OS = Acidaminococcus sp. (strain BV3L6) OX = 1111120 GN = cas12a PE = 1 SV = 1
SEQ ID NO: 8
>tr|A0A5S8WF58|A0A5S8WF58 9FIRM LbCas12a OS = Lachnospiraceae bacterium
OX = 1898203 PE = 1 SV = 1
SEQ ID NO: 9
SaCAS9_KKH (E782K/N968K/R1015H)
SEQ ID NO: 10
cCAS9_v42
SEQ ID NO: 11
cCAS9_v17
SEQ ID NO: 12
cCAS9_v16
SEQ ID NO: 13
cCAS9_v21
SEQ ID NO: 14
SaCAS9_N986R
SEQ ID NO: 15
SaCAS9_N986R+R991L
SEQ ID NO: 16
SaCAS9_DNA sequence
SEQ ID NO: 17
SpCas9_DNA sequence
SEQ ID NO: 18
>sp A0Q5Y3|CAS9_FRATN CRISPR-associated endonuclease Cas9
OS = Francisella tularensis subsp. novicida (strain U112) OX = 401614
GN = CAS9_PE = 1 SV = 1
SEQ ID NO: 19
>tr|Q5M542 Q5M542 STRT2 CRISPR-associated endonuclease Cas9
OS = Streptococcus thermophilus (strain ATCC BAA-250/LMG 18311)
OX = 264199 GN = CAS9_PE = 3 SV = 1
SEQ ID NO: 20
>sp|C9X1G5|CAS9_NEIM8 CRISPR-associated endonuclease CAS9_OS = Neisseria
meningitidis serogroup C (strain 8013) OX = 604162 GN = CAS9_PE = 1 SV = 1
SEQ ID NO: 21
>sp|Q0P897|CAS9_CAMJE CRISPR-associated endonuclease Cas9
OS = Campylobacter jejuni subsp. jejuni serotype 0:2 (strain ATCC 700819
/NCTC 11168) OX = 192222 GN = CAS9_PE = 1 SV = 1
SEQ ID NO: 22
sp|U2UMQ6|CS12A ACISB CRISPR-associated endonuclease Cas12a
OS = Acidaminococcus sp. (strain BV3L6) OX = 1111120 GN = cas12a PE = 1 SV = 1
SEQ ID NO: 23
>tr|A0A5S8WF58|A0A5S8WF58 9FIRM LbCas12a OS = Lachnospiraceae bacterium
OX = 1898203 PE = 1 SV = 1
SEQ ID NO: 24
SaCAS9_Y256A_R314A
SEQ ID NO: 25
SaCAS9_Y256A_R314A_Q414A
SEQ ID NO: 26
SaCas9_Y256A_R314A_R654A
SEQ ID NO: 27
SaCAS9_Y256A_R314A_Q414A_R654A
SEQ ID NO: 28
SpCAS9_F518A_E579A (aa)
SEQ ID NO: 29
SpCAS9_F518A_E579A_G687A (aa)
SEQ ID NO: 30
SpCAS9_F518A_E579A_K929A (aa)
SEQ ID NO: 31
SpCAS9_F518A_E579A_G687A_K929A (aa)
SEQ ID NO: 32
SaCAS9_Y256A_R314A (nt)
SEQ ID NO: 33
SaCAS9_Y256A_R314A_Q414A
SEQ ID NO: 34
SaCAS9_Y256A_R314A_R654A (nt)
SEQ ID NO: 35
SaCAS9_Y256A_R314A_Q414A R654A
SEQ ID NO: 36
SpCAS9_F518A_E579A (nt)
SEQ ID NO: 37
SpCAS9_F518A_E579A_G687A (nt)
SEQ ID NO: 38
SpCAS9_F518A_E579A_K929A (nt)
SEQ ID NO: 39
SpCAS9_F518A_E579A_G687A_K929A (nt)

1. A CRISPR polypeptide, wherein the amino acid sequence of the CRISPR polypeptide has:
(a) at least 90% amino acid sequence identity with one or more of the reference sequences given in SEQ ID NOs: 1-15; and
(b) wherein, for each reference sequence to which the CRISPR polypeptide has at least 90% sequence identity,
(i) the amino acid in the CRISPR polypeptide at the position which corresponds to position Y256 in SEQ ID NO: 1 is an aliphatic amino acid; and
(ii) the amino acid in the CRISPR polypeptide at the position which corresponds to position R314 in SEQ ID NO: 1 is an aliphatic amino acid

2. (canceled)

3. (canceled)

4. The CRISPR polypeptide as claimed in claim 1, wherein additionally:
(c) the amino acid in the CRISPR polypeptide at the position which corresponds to position Q414 in SEQ ID NO: 1 is an aliphatic amino acid.

5. The CRISPR polypeptide as claimed in claim 1, wherein additionally:
(d) the amino acid in the CRISPR polypeptide at the position which corresponds to position R654 in SEQ ID NO: 1 is an aliphatic amino acid.

6. The CRISPR polypeptide as claimed in claim 1, wherein the reference sequence is selected from the group consisting of:
(A) SEQ ID NOs: 1-8; or
(B) SEQ ID NO: 1 and SEQ ID NO: 2.

7. (canceled)

8. The CRISPR polypeptide as claimed in claim 1, wherein the aliphatic amino acids are independently selected from the group consisting of alanine, leucine, isoleucine and valine.

9. The CRISPR polypeptide as claimed in claim 1, wherein one or more or all of the aliphatic amino acids are alanine.

10. The CRISPR polypeptide as claimed in claim 1, wherein the CRISPR polypeptide is a Cas9, a Cas12 or a Cas13 polypeptide.

11. A Cas9 polypeptide, wherein the amino acid sequence of the polypeptide is given in or comprises any one of SEQ ID NOs: 24-31.

12. (canceled)

13. (canceled)

14. (canceled)

15. A Cas9 polypeptide, wherein the amino acid sequence of the Cas9 polypeptide has:
(i) at least 90% amino acid sequence identity with SEQ ID NO: 2; and
(ii) mutations at positions corresponding to positions F518 and E579 in SEQ ID NO: 2,
wherein the mutations are single amino acid substitutions, and wherein the substitutions are for an aliphatic amino acids or for alanine.

16. The Cas9 polypeptide as claimed in claim 15, wherein the amino acid sequence of the Cas9 polypeptide additionally has:
(iii) a mutation at the position corresponding to position G687 in SEQ ID NO: 2.

17. The Cas9 polypeptide as claimed in claim 15, wherein the amino acid sequence of the Cas9 polypeptide additionally has:
(iv) a mutation at the position corresponding to position K929 in SEQ ID NO: 2.

18. (canceled)

19. The CRISPR polypeptide as claimed in claim 1, wherein the CRISPR polypeptide:
(i) has nuclease or endonuclease activity; or
(ii) is nuclease-deficient, or is a dCas9 or a dCas12.

20. The CRISPR polypeptide as claimed in claim 1, wherein the CRISPR polypeptide:
(i) is a CRISPR activator (CRISPRa) polypeptide; or
(ii) is a CRISPR inhibitor (CRISPRi) polypeptide.

21. The CRISPR polypeptide as claimed in claim 1, wherein
(A) the CRISPR polypeptide is fused to a DNA-modifying domain or a heterologous functional domain or an effector domain; or
(B) the CRISPR polypeptide is fused to a deaminase and/or a reverse transcriptase.

22. (canceled)

23. A nucleic acid molecule encoding a CRISPR polypeptide as claimed in claim 1, or a nucleic acid molecule comprising a nucleotide sequence selected from the group consisting of SEQ ID NOs: 32-39.

24. A vector or plasmid comprising a nucleic acid molecule as claimed in claim 23.

25. A host cell comprising
a nucleic acid molecule as claimed in claim 23.

26. A kit comprising:
(i) a CRISPR polypeptidee as claimed in claim 1; and
(ii) a guide RNA.

27. A method of targeting and/or modifying a target DNA molecule, the method comprising contacting:
(i) a CRISPR polypeptideas claimed in claim 1; and
(ii) a guide RNA having a region complementary to the target DNA molecule, with the target DNA molecule.
